(19) United States
(12) Patent Application Publication
(10) Pub. No.: US 2025/0308023 A1
(43) Pub. Date: Oct. 2, 2025

(54) DETECTING AND QUANTIFYING HYPERREFLECTIVE FOCI (HRF) IN RETINAL PATIENTS

(71) Applicant: Hoffmann-La Roche Inc., Little Falls, NJ (US)

(72) Inventors: Huanxiang LU, Préverenges (CH); Andreas MAUNZ, Freiburg (DE); Yaniv COHEN, Riehen (CH); Ian Lloyd JONES, Basel (CH)

(21) Appl. No.: 19/238,111

(22) Filed: Jun. 13, 2025

Related U.S. Application Data
(63) Continuation of application No. PCT/US2023/083890, filed on Dec. 13, 2023.
(60) Provisional application No. 63/432,654, filed on Dec. 14, 2022.

(51) Int. Cl. G06T 7/00; G16H 20/10

(52) U.S. Cl. CPC G06T 7/0012; G16H 20/10; G06T 2207/10101; G06T 2207/20081; G06T 2207/20084; G06T 2207/30041

(57) ABSTRACT

A method for identifying hyperreflective foci (HRF) in an eye of a patient includes accessing one or more optical coherence tomography (OCT) scans of a retina of the eye of the patient, and inputting the one or more OCT scans into one or more machine-learning models trained to segment the one or more OCT scans to identify a set of hyperreflective entities detectable from the one or more OCT scans. The method further includes determining, based on the segmented one or more OCT scans, one or more diametral measurements corresponding to each of the identified set of hyperreflective entities, and identifying hyperreflective foci (HRF) in the retina of the eye of the patient based on whether at least one of the one or more diametral measurements satisfy a diametral threshold.

### CROSS-REFERENCE TO RELATED APPLICATIONS

This application is a continuation of International Application No. PCT/US2023/083890, filed on Dec. 13, 2023, which claims priority to U.S. Provisional Patent Application No. 63/432,654, filed Dec. 14, 2022, entitled “DETECTING AND QUANTIFYING HYPERREFLECTIVE FOCI (HRF) IN RETINAL PATIENTS,” the content of which is hereby incorporated by reference in its entirety.

### TECHNICAL FIELD

This application generally relates to hyperreflective foci (HRF), and, more particularly, to detecting and quantifying HRF in the retina of an eye of a patient.

### BACKGROUND

Hyperreflective foci (HRF) have been shown to be associated with various retinal diseases, such as diabetic retinopathy (DR), diabetic macular edema (DME), age-related macular degeneration (AMD), neovascular age-related macular degeneration (nAMD), geographic atrophy (GA), macular atrophy (MA), and retinal vein occlusion (RVO). Indeed, recent research of HRF has demonstrated that these lesions may represent important biomarkers for retinal disease progression, prognosis, and treatment outcomes. HRF may include discrete, well-circumscribed lesions characterized by reflectivity equal to or greater than the retinal pigment epithelium (RPE). HRF may be identified in some optical coherence tomography (OCT) scans. However, quantification and accurate identification of HRF remains a significant challenge to advancing the understanding of the role HRF plays in the pathogenesis of the aforementioned retinal diseases.

Specifically, computational-based segmentation of HRF remains a challenging and elusive task. For example, many existing computational-based models struggle to distinguish HRF, for example, from retinal blood vessels, hard exudates, and speckle noise, particularly when accompanied by the presence of other retinal disease biomarkers. Indeed, while computational-based segmentation of HRF may ostensibly improve the ability to identify and study HRF, there remains significant challenges to developing and deploying computational-based segmentation models in clinical practice. Such challenges include a lack of adequate training data, as manual annotation of HRF or other similar hyperreflective materials may be time-consuming, costly, and susceptible to immense human error. Such challenges may further include the lack of a unifying standard for defining and accurately identifying HRF, as many existing computational-based models may identify any material (e.g., hard exudates, generic hyperreflective material (HRM), speckle noise) less than 100 microns (μm) as HRF. It may be thus useful to provide techniques to accurately detect and quantify HRF in the retina of an eye of a patient.

### SUMMARY

Embodiments of the present disclosure are directed toward one or more computing devices, methods, and non-transitory computer-readable media for detecting and quantifying hyperreflective foci (HRF) in the retina of an eye of a patient. In certain embodiments, one or more computing devices may access one or more optical coherence tomography (OCT) B-scans of a retina of an eye of a patient. In certain embodiments, the one or more computing devices may then input the one or more OCT B-scans into one or more machine-learning models (e.g., semantic segmentation model) of an HRF segmentation and classification pipeline, in which the one or more machine-learning models (e.g., semantic segmentation model) may be trained to segment the one or more OCT B-scans to identify a set of hyperreflective entities detectable from the one or more OCT B-scans. For example, in some embodiments, the one or more machine-learning models (e.g., semantic segmentation model) may generate a prediction of a segmentation map, in which the segmentation map identifies the set of hyperreflective entities. In some embodiments, the identified set of hyperreflective entities may include various hyperreflective entities, including, for example, hyperreflective material (HRM), intraretinal hyperreflective material (IHRM), and HRF.

In certain embodiments, the one or more machine-learning models (e.g., semantic segmentation model) may then output the identified set of hyperreflective entities (e.g., HRM, IHRM, HRF) to a classification module (e.g., image-processing-based algorithm) of the HRF segmentation and classification pipeline. The classification module (e.g., image-processing-based algorithm) may then be utilized to determine one or more diametral measurements corresponding to each of the identified set of hyperreflective entities, and further to identify HRF in the retina of the eye of the patient based on whether at least one of the one or more diametral measurements satisfy a diametral threshold.

Specifically, in accordance with the presently disclosed embodiments, the classification module (e.g., image-processing-based algorithm) may estimate the diameter of each of the identified set of hyperreflective entities (e.g., HRM, IHRM, HRF) and classify each hyperreflective entity having an estimated diameter of 50 microns (μm) or less as HRF and classify each hyperreflective entity having an estimated diameter within a range of 50 μm to 100 μm as IHRM. In some embodiments, the classification module can further classify each hyperreflective entity having an estimated diameter above 100 μm as belonging to a third class of objects. In certain embodiments, a feature extraction module (e.g., image-processing-based algorithm) of the HRF segmentation and classification pipeline, based on mapping information of the Early Treatment for Diabetic Retinopathy Study (ETDRS) grid, may be then utilized to calculate one or more volumetric measurements (e.g., a volume, an area, a thickness, a quantity, and so forth) of the identified HRF within one or more corresponding ETDRS subfields.

In this way, the disclosed embodiments may provide an HRF segmentation and classification pipeline that may be suitable for accurately detecting and quantifying HRF in the retina of an eye of a patient, in which HRF is specifically defined as a hyperreflective entity having an estimated diameter of 50 μm or less. Indeed, by providing a HRF segmentation and classification pipeline suitable for accurately detecting and quantifying HRF in the retina of an eye of a patient, the present embodiments accurately and efficiently identify a clinically-significant biomarker of visual acuity and morphological changes in many retinal diseases, such as diabetic retinopathy (DR), diabetic macular edema (DME), age-related macular degeneration (AMD), neovascular age-related macular degeneration (nAMD), geographic atrophy (GA), macular atrophy (MA), and retinal vein occlusion (RVO). Additionally, the provided HRF segmentation and classification pipeline may be further suitable for utilizing the detected and quantified HRF to predict retinal disease progression and patient treatment response in accordance with the presently disclosed embodiments.

In certain embodiments, the one or more computing devices may access one or more optical coherence tomography (OCT) scans of a retina of an eye of a patient. In certain embodiments, the one or more computing devices may input the one or more OCT scans into one or more machine-learning models trained to segment the one or more OCT scans to identify a set of hyperreflective entities detectable from the one or more OCT scans. In certain embodiments, the set of hyperreflective entities may include a set of a hyperreflective material (HRM), intraretinal hyperreflective material (IHRM), and HRF. In certain embodiments, the one or more machine-learning models may include at least one semantic segmentation model. In one embodiment, the at least one semantic segmentation model may include a U-Net architecture.

In certain embodiments, the one or more computing devices may then determine, based on the segmented one or more OCT scans, one or more diametral measurements corresponding to each of the identified set of hyperreflective entities. In certain embodiments, the one or more computing devices may then identify HRF in the retina of the eye of the patient based on whether at least one of the one or more diametral measurements satisfy a diametral threshold. For example, in some embodiments, determining whether the at least one of the one or more diametral measurements satisfy the diametral threshold may include, for each of the identified set of hyperreflective entities, associating an ellipse with the identified hyperreflective entity, determining a diameter of a longest axis of the ellipse, and estimating, based on the diameter of the longest axis of the ellipse, the at least one of the one or more diametral measurements.

In certain embodiments, the one or more computing devices may identify HRF in the retina of the eye of the patient by identifying a subset of the identified set of hyperreflective entities. For example, in one embodiment, the diametral threshold may include a minimum diameter of approximately 50 microns (μm). In certain embodiments, the one or more computing devices may identify intraretinal hyperreflective material (IHRM) in the retina of the eye of the patient based on whether the at least one of the one or more diametral measurements satisfy a second diametral threshold. For example, in one embodiment, the second diametral threshold comprises a diameter range of approximately 50 μm to 100 μm. In some embodiments, the classification module can further classify each hyperreflective entity having an estimated diameter above 100 μm as belonging to a third class of objects. In certain embodiments, identifying HRF in the retina of the eye of the patient further may include classifying the eye of the patient as having at least one of diabetic retinopathy (DR), diabetic macular edema (DME), age-related macular degeneration (AMD), neovascular age-related macular degeneration (nAMD), geographic atrophy (GA), macular atrophy (MA), or retinal vein occlusion (RVO).

In certain embodiments, identifying HRF in the retina of the eye of the patient may further include accessing Early Treatment for Diabetic Retinopathy Study (ETDRS) grid mapping information identifying one or more subfields of the ETDRS grid, and determining, based at least in part on the ETDRS grid mapping information, one or more volumetric measurements of the identified HRF. In some embodiments, the one or more volumetric measurements may include one or more of a volume of HRF in the retina of the eye of the patient, an area of HRF in the retina of the eye of the patient, or a thickness of HRF in the retina of the eye of the patient.

For example, in some embodiments, the one or more computing devices may determine, based at least in part on the ETDRS grid mapping information, a volume of HRF in the retina of the eye of the patient with respect to at least one of the identified one or more subfields. In one embodiment, determining the volume of HRF in the retina of the eye of the patient may include determining a reduction in the volume of HRF in the retina of the eye of the patient. In one embodiment, the at least one of the identified one or more subfields may include an outer retina subfield of the ETDRS grid.

In certain embodiments, the one or more computing devices may determine, based at least in part on the ETDRS grid mapping information, a quantity of HRF in the retina of the eye of the patient with respect to at least one of the identified one or more subfields. In certain embodiments, the one or more computing devices may access an en face image of the retina of the eye of the patient, in which the en face image is associated with the one or more OCT scans. In certain embodiments, the one or more computing devices may then map, based at least in part on the ETDRS grid mapping information, the identified HRF to the en face image.

In certain embodiments, the one or more computing devices may train the one or more machine-learning models. For example, in some embodiments, training the one or more computing devices may include accessing a data set of OCT scans of a retina of an eye of one or more patients. For example, in one embodiment, the data set of OCT scans may include sparse annotations of HRF in the retina of the eye of the one or more patients. In certain embodiments, training the one or more computing devices may further include partitioning the data set of OCT scans into a model-training data set and a model-validation data set, training, based on the model-training data set, the one or more machine-learning models to segment OCT scans to identify sets of hyperreflective entities detectable from the OCT scans. In one embodiment, the identified sets of hyperreflective entities may include hyperreflective material (HRM).

In certain embodiments, training the one or more computing devices may further include evaluating the one or more machine-learning models based on the model-validation data set. In certain embodiments, training the one or more computing devices may further include identifying HRF in the retina of the eye of the one or more patients based on whether one or more diametral measurements of the HRM satisfy a predetermined diametral threshold. In certain embodiments, the sparse annotations of HRF may include a bounding geometry encompassing a plurality of instances of HRF, and thus training the one or more computing devices may further include performing an adjustment of the bounding geometry by reducing a size of the bounding geometry, in which the size of the bounding geometry being is reduced to annotate a single instance of HRF.

In certain embodiments, the one or more OCT scans may include one or more first OCT scans of the retina of the eye of the patient being captured at an initial date. In one embodiment, the identified HRF may include a first volume of HRF. In certain embodiments, the one or more computing devices may access one or more second OCT scans of the retina of the eye of the patient. In certain embodiments, the one or more computing devices may then input the one or more second OCT scans into the one or more machine-learning models to segment the one or more second OCT scans to identify a second set of hyperreflective entities detectable from the one or more second OCT scans. In certain embodiments, the one or more computing devices may then determine, based on the segmented one or more second OCT scans, one or more second diametral measurements corresponding to each of the identified second set of hyperreflective entities.

In certain embodiments, the one or more computing devices may then identify a second volume of HRF in the retina of the eye of the patient based on whether at least one of the one or more second diametral measurements satisfy the diametral threshold. In one embodiment, the one or more computing devices may then determine, based on the second volume of HRF, whether the eye of the patient is responsive to a treatment. In another embodiment, the one or more computing devices may further determine, based on the second volume of HRF, a degree to which the eye of the patient is responsive to the treatment. For example, in some embodiments, the eye of the patient is responsive to the treatment when the second volume of HRF is less than the first volume of HRF. In certain embodiments, the one or more second OCT scans are captured at one or more dates selected from the group comprising approximately 0.25 months, 0.5 months, 0.75 months, 1 month, 3 months, 6 months, 9 months, 12 months, 15 months, 18 months, 21 months, 24 months, 27 months, 30 months, 33 months, or 36 months from the initial date.

In certain embodiments, the treatment may include an anti-vascular endothelial growth factor (anti-VEGF) antibody, an anti-vascular endothelial growth factor-A (anti-VEGF-A) antibody, an anti-anangiopoietin-2 (anti-Ang-2) antibody, or a combination thereof. In certain embodiments, the anti-VEGF-A antibody may include faricimab-svoa. In certain embodiments, the anti-Ang-2 antibody may include faricimab-svoa. In certain embodiments, the anti-VEGF antibody may be selected from the group including ranibizumab, aflibercept, brolucizumab, bevacizumab, and pegaptanib sodium. In certain embodiments, the one or more computing devices may identify an effective treatment regimen of an anti-vascular endothelial growth factor (anti-VEGF) antibody, an anti-anangiopoietin-2 (anti-Ang-2) antibody, or a combination thereof, to treat the eye of the patient based on a volume or a quantity of the identified HRF.

For example, in one embodiment, identifying the effective treatment regimen may include identifying, based on the volume or the quantity of the identified HRF, a dosage for administering the anti-VEGF antibody, the anti-Ang-2 antibody, or the combination thereof. In another embodiment, identifying the effective treatment regimen may include identifying, based on the volume or the quantity of the identified HRF, a schedule for administering the anti-VEGF antibody, the anti-Ang-2 antibody, or the combination thereof. In another embodiment, identifying the effective treatment regimen may include identifying, based on the volume or the quantity of the identified HRF, a duration for administering the anti-VEGF antibody, the anti-Ang-2 antibody, or the combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a retinal segmentation, classification, and feature extraction network and system in accordance with some embodiments disclosed herein.

FIG. 2A illustrates a diagram of an inference phase of an HRF segmentation and classification pipeline suitable for detecting and quantifying HRF in the retina of an eye of a patient in accordance with some embodiments disclosed herein.

FIG. 2B illustrates a diagram of a training phase of an HRF segmentation and classification pipeline suitable for detecting and quantifying HRF in the retina of an eye of a patient in accordance with some embodiments disclosed herein.

FIG. 2C illustrates a diagram of a classification stage of an HRF segmentation and classification pipeline in accordance with some embodiments disclosed herein.

FIG. 3A illustrates a flow diagram of a method for identifying hyperreflective foci (HRF) in an eye of a patient in accordance with some embodiments disclosed herein.

FIG. 3B illustrates a flow diagram of a method for determining, based on a change in volume or quantity of HRF, whether an eye of a patient is responsive to a treatment in accordance with some embodiments disclosed herein.

FIG. 4A illustrates an enlarged example image of an OCT B-scan in accordance with some embodiments disclosed herein.

FIG. 4B illustrates an enlarged example image of a segmented and classified OCT B-scan in accordance with some embodiments disclosed herein.

FIGS. 5A and 5B illustrate enlarged example images of a first en face image and a second en face image, respectively in accordance with some embodiments disclosed herein.

FIG. 6 illustrates an example computing system in accordance with some embodiments disclosed herein.

FIG. 7 illustrates a diagram of an example artificial intelligence (AI) architecture included as part of the example computing system of FIG. 6 in accordance with some embodiments disclosed herein.

### DESCRIPTION OF EXAMPLE EMBODIMENTS

Embodiments of the present disclosure are directed toward one or more computing devices, methods, and non-transitory computer-readable media for detecting and quantifying hyperreflective foci (HRF) in the retina of an eye of a patient. In certain embodiments, one or more computing devices may access one or more optical coherence tomography (OCT) B-scans of a retina of an eye of a patient. In certain embodiments, the one or more computing devices may then input the one or more OCT B-scans into one or more machine-learning models (e.g., semantic segmentation model) of an HRF segmentation and classification pipeline, in which the one or more machine-learning models (e.g., semantic segmentation model) may be trained to segment the one or more OCT B-scans to identify a set of hyperreflective entities detectable from the one or more OCT B-scans. For example, in some embodiments, the one or more machine-learning models (e.g., semantic segmentation model) may generate a prediction of a segmentation map, in which the segmentation map identifies the set of hyperreflective entities. In some embodiments, the identified set of hyperreflective entities may include various hyperreflective entities, including, for example, hyperreflective material (HRM), intraretinal hyperreflective material (IHRM), and HRF.

In certain embodiments, the one or more machine-learning models (e.g., semantic segmentation model) may then output the identified set of hyperreflective entities (e.g., HRM, IHRM, HRF) to a classification module (e.g., image-processing-based algorithm) of the HRF segmentation and classification pipeline. The classification module (e.g., image-processing-based algorithm) may then be utilized to determine one or more diametral measurements corresponding to each of the identified set of hyperreflective entities, and further to identify HRF in the retina of the eye of the patient based on whether at least one of the one or more diametral measurements satisfy a diametral threshold.

Specifically, in accordance with the presently disclosed embodiments, the classification module (e.g., image-processing-based algorithm) may estimate the diameter of each of the identified set of hyperreflective entities (e.g., HRM, IHRM, HRF) and classify each hyperreflective entity having an estimated diameter of 50 microns (μm) or less as HRF and classify each hyperreflective entity having an estimated diameter within a range of 50 μm to 100 μm as IHRM. In some embodiments, the classification module can further classify each hyperreflective entity having an estimated diameter above 100 μm as belonging to a third class of objects. In certain embodiments, a feature extraction module (e.g., image-processing-based algorithm) of the HRF segmentation and classification pipeline, based on mapping information of the Early Treatment for Diabetic Retinopathy Study (ETDRS) grid, may be then utilized to calculate one or more volumetric measurements (e.g., a volume, an area, a thickness, a quantity, and so forth) of the identified HRF within one or more corresponding ETDRS subfields.

In this way, the disclosed embodiments may provide an HRF segmentation and classification pipeline that may be suitable for accurately detecting and quantifying HRF in the retina of an eye of a patient, in which HRF is specifically defined as a hyperreflective entity having an estimated diameter of 50 μm or less. Indeed, by providing a HRF segmentation and classification pipeline suitable for accurately detecting and quantifying HRF in the retina of an eye of a patient, the present embodiments accurately and efficiently identify a clinically-significant biomarker of visual acuity and morphological changes in many retinal diseases, such as diabetic retinopathy (DR), diabetic macular edema (DME), age-related macular degeneration (AMD), neovascular age-related macular degeneration (nAMD), geographic atrophy (GA), macular atrophy (MA), and retinal vein occlusion (RVO). Additionally, the provided HRF segmentation and classification pipeline may be further suitable for utilizing the detected and quantified HRF to predict retinal disease progression and patient treatment response in accordance with the presently disclosed embodiments.

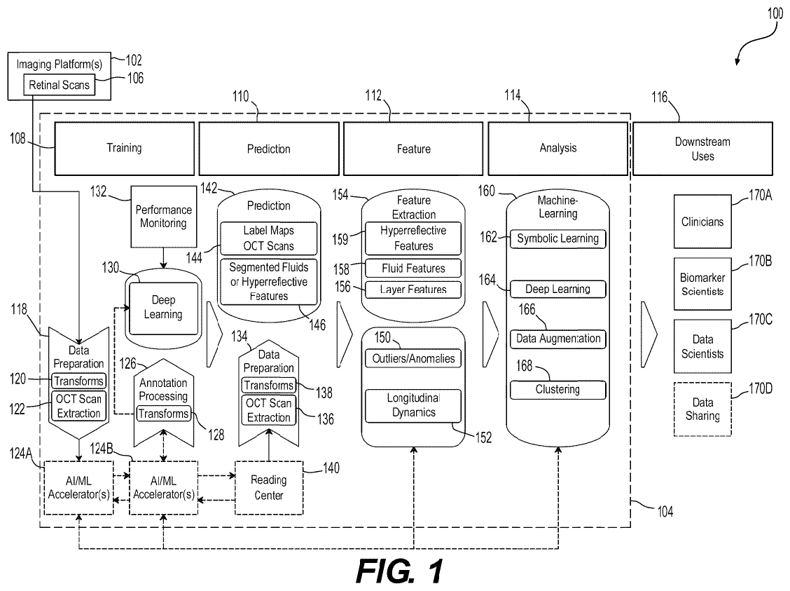

FIG. 1 illustrates a retinal segmentation, classification, and feature extraction system 100 that may be utilized for detecting and quantifying HRF in the retina of an eye of a patient, in accordance with the presently disclosed embodiments. In certain embodiments, the retinal segmentation, classification, and feature extraction system 100 may include one or more retinal imaging platforms 102 and a retinal segmentation, classification, and feature extraction pipeline 104. For example, in some embodiments, the one or more imaging platforms 102 may include one or more non-invasive retinal scan-capturing devices (e.g., ophthalmoscope, scanning laser, ultra-wide field fundus camera, or other retinal scan-capturing module), which may scan a patient's retina and generate one or more high-resolution, 2D or 3D retinal scans 106.

For example, in some embodiments, the retinal scans 106 may include one or more OCT scans (e.g., time-domain-OCT (TD-OCT) scan, spectral-domain-OCT (SD-OCT) scan), which may be used to capture and render retinal layer depth. Specifically, in certain embodiments, in capturing an image of the patient's retina, the one or more retinal imaging platforms 102 may perform a series of one-dimensional (1D) scans (e.g., amplitude scan or “A-scan”) at different depths or positions and generate a 2D, cross-sectional image (e.g., brightness scan or “B-scan”) of the patient's three-dimensional (3D) retina utilizing the series of OCT A-scans. In certain embodiments, by closely and rapidly acquiring and generating the OCT B-scans, the one or more imaging platforms 102 may further generate one or more volumetric images (“C-scans”) of the patient's three-dimensional (3D) retina.

In other embodiments, the retinal scans 106 may include one or more color fundus photography (CFP) images (e.g., multicolor 2D image of the retina, infrared (IR) 2D image of the retina), one or more retinal angiography scans (e.g., fluorescein angiography (FA) scan, OCT-angiography (OCT-A) scan, ultra-wide field fluorescein angiography (UWFA) scan) images (e.g.,), an ultra-wide field fluorescein angiography (UWFA) scan, indocyanine green angiography (ICGA)) scan), one or more fundus autofluorescence (FAF) scans, one or more blue light autofluorescence (BAF) scans, other similar retinal scan. In one embodiment, the retinal scans 106 (e.g., a number of OCT B-scans) may be captured by a retinal specialist (e.g., ophthalmologist, optometrist) during one or more visits and one or more subsequent visits by a patient to a clinical setting. In another embodiment, the retinal scans 106 (e.g., a number of OCT B-scans) may include a data set of retinal scans (e.g., OCT B-scans) captured from various patients during one or more clinical trials. In one embodiment, the data set of retinal scans (e.g., OCT B-scans) may be annotated and utilized to train one or more segmentation, classification, and feature extraction machine-learning (ML) models or similar models.

In certain embodiments, as further depicted by FIG. 1, the one or more retinal imaging platforms 102 may then provide the retinal scans 106 (e.g., a number of OCT B-scans) to the retinal segmentation, classification, and feature extraction pipeline 104. In certain embodiments, the retinal segmentation, classification, and feature extraction pipeline 104 may include training module and submodules 108, prediction module and submodules 110, feature module and submodules 112, and analysis module and submodules 114 that may be utilized in one or more downstream uses 116. While the training and execution of the training module and submodules 108, the prediction module and submodules 110, the feature module and submodules 112, and the analysis module and submodules 114 may be discussed herein in a generally sequential manner (e.g., for the purposes of conciseness and illustration), it should be appreciated that the training module and submodules 108, the prediction module and submodules 110, the feature module and submodules 112, and the analysis module and submodules 114 may be trained and/or executed according to an end-to-end deep learning process. For example, in certain embodiments, the training module and submodules 108, the prediction module and submodules 110, the feature module and submodules 112, and the analysis module and submodules 114 may be trained and/or executed end-to-end (e.g., preprocessing, feature extraction and selection, optimization, prediction, decision making, and so forth) as a single, ensemble model for\detecting and quantifying HRF in the retina of an eye of a patient.

In view of the foregoing, in certain embodiments, during the training phase, the retinal scans 106 (e.g., a number of OCT B-scans) may be provided to a data processing functional block 118. In certain embodiments, the data processing functional block 118 may estimate or determine the quality of the retinal scans 106 (e.g., a number of OCT B-scans) and perform one or more transforms 120 and OCT B-scans extractions 122 to improve and/or enhance the quality of the retinal scans 106 (e.g., a number of OCT B-scans) or one or more features included within the retinal scans 106 (e.g., a number of OCT B-scans) based on the estimation of the quality. For example, the retinal scans 106 may include a number of OCT B-scans extractions 122 of the retina of one or more patients that may be preprocessed to improve and/or enhance, for example, image quality, local contrast, image resolution, speckle noise, and so forth. Similarly, the one or more transforms 120 may include one or more transforms (e.g., super-resolution algorithms, image-alignment algorithms, pixel-alignment algorithms, image-stitching, and so forth) to further improve and/or enhance the quality of the retinal scans 106 (e.g., a number of OCT B-scans).

In certain embodiments, as further depicted by FIG. 1, the retinal segmentation, classification, and feature extraction pipeline 104 may include one or more artificial intelligence (AI)/machine-learning (ML) accelerators 124A, 124B (e.g., one or more of a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU)) that may be suitable for hosting, executing, and/or processing the various training module and submodules 108, prediction module and submodules 110, feature module and submodules 112, and/or analysis module and submodules 114.

In certain embodiments, following the transforms 120 and OCT B-scan extractions 122 as performed by the data processing functional block 118, the retinal scans 106 (e.g., a number of OCT B-scans) may be provided to an annotation processing functional block 126. For example, in some embodiments, the retinal scans 106 (e.g., a number of OCT B-scans) may include a data set of retinal scans (e.g., a number of OCT B-scans) that may be pre-annotated or sparsely annotated, for example, and utilized to train one or more deep learning models 130 for performing semantic segmentation (e.g., pixel-wise segmentation) and classification to segment and annotate one or more layer features (e.g., layers of the retina), one or more fluid features, or one or more hyperreflective entities detectable from the retinal scans 106 (e.g., a number of OCT B-scans).

For example, in certain embodiments, the one or more deep learning models 130 may include a deep residual neural network (ResNet) image-classification network (e.g., ResNet-34, ResNet-50, ResNet-101, ResNet-152), a full-resolution residual network (FRRN), a fully convolutional network (FCN) (e.g., U-Net), a pyramid scene parsing network (PSPNet), a fully convolutional dense neural network (FCDenseNet), a multi-path refinement network (RefineNet), an atrous convolutional network (e.g., DeepLabV3, DeepLabV+), a semantic segmentation network (SegNet), or other deep convolutional neural network (DCNN) that may be suitable for performing semantic segmentation and classification to segment and annotate one or more layer features (e.g., layers of the retina), one or more fluid features, or hyperreflective entities detectable from the retinal scans 106 (e.g., a number of OCT B-scans). In one embodiment, a performance monitoring functional block 132 may be provided to monitor and evaluate the one or more deep learning models 130 during the training phase until the one or more deep learning models 130 are sufficiently trained.

In certain embodiments, after the one or more deep learning models 130 are sufficiently trained for performing, for example, semantic segmentation (e.g., pixel-wise segmentation) and classification to segment and annotate one or more layer features (e.g., layers of the retina), one or more fluid features, or one or more hyperreflective entities detectable from the retinal scans 106 (e.g., a number of OCT B-scans), during the inference phase, the retinal scans 106 (e.g., a number of OCT B-scans) may be provided to a data processing functional block 134. The data processing functional block 134 may perform one or more transforms 136 and OCT B-scan extractions 138. For example, the retinal scans 106 may include a number of OCT B-scan extractions 138 of the retina of one or more patients that may be preprocessed to improve, for example, image quality, local contrast, image resolution, speckle noise, and so forth. Similarly, the one or more transforms 136 may include one or more transforms (e.g., super-resolution algorithms, image-alignment algorithms, pixel-alignment algorithms, image-stitching, and so forth) to further improve the quality of the retinal scans 106 (e.g., a number of OCT B-scans).

In certain embodiments, prior to providing the preprocessed retinal scans 106 (e.g., a number of OCT B-scans) to a prediction functional block 142, one or more fluid features or hyperreflective entities of at least a subset of the retinal scans 106 (e.g., a number of B-scans) may be annotated by way of reading center data inputs 140. In some embodiments, the reading center data inputs 140 may include annotations performed by one or more medical or scientific experts, for example, from an ophthalmology reading center. For example, in one embodiment, the retinal scans 106 (e.g., a number of OCT B-scans) may be annotated manually by drawing bounding geometries or contours, for example, representative of fluid features or hyperreflective entities.

In certain embodiments, pixel regions and corresponding labels of the layers of the retina and the fluid features or hyperreflective entities may be predicted utilizing the one or more deep learning models 130. For example, the one or more deep learning models 130 may generate predictions of one or more label maps 144 (e.g., including one or more OCT B-scans with the layers of the retina segmented and annotated) and segmented fluid features or hyperreflective entities 146 (e.g., fluid features and/or hyperreflective entities segmented and annotated on the one or more OCT B-scans).

In some embodiments, the layers of the retina may include one or more of a Bruch's membrane (BM), a boundary of myoid and ellipse inner segments (BMEIS), a ganglion cell layer-inner plexiform layer (GCL-IPL), an inner boundary outer photoreceptor (IB-OPR) layer, an outer boundary outer photoreceptor (OB-OPR) layer, an inner boundary retinal pigment epithelium (IB-RPE) layer, an outer boundary retinal pigment epithelium (OB-RPE) layer, an internal limiting membrane (ILM), an inner plexiform layer-inner nuclear layer (IPL-INL), an inner plexiform layer-outer nuclear layer (IPL-ONL), an inner segment/outer segment junction (ISJ-OSJ) layer, outer plexiform layer-Henle's fiber layer (OPL-HFL), or an retinal nerve fiber layer-ganglion cell layer (RNFL-GCL). Similarly, in some embodiments, the fluid features may include, one or more of an intraretinal fluid (IRF), a subretinal fluid (SRF), a pigment epithelial detachment (PED), or subretinal hyperreflective material (SHRM). In certain embodiments, the hyperreflective entities may include a hyperreflective material (HRM), an intraretinal hyperreflective material (IHRM), or hyperreflective foci (HRF).

In certain embodiments, following the generation of the predictions of the one or more label maps 144 (e.g., including one or more OCT B-scans with the layers of the retina segmented and annotated) and segmented fluid features or hyperreflective features 146 (e.g., IRF, SRF, PED, SHRM, IHRM, HRF segmented and labeled on the one or more OCT B-scans), the one or more label maps 144 and the segmented fluid features or hyperreflective entities 146 may be provided to a feature calculation functional block 154. In certain embodiments, the feature calculation functional block 154 may be utilized to extract one or more volumetric measurements of layer features 156, fluid features 158, and hyperreflective entities 159.

For example, in certain embodiments, the one or more volumetric measurements of layer features 156, fluid features 158, and hyperreflective entities 159 may include a matrix of volume, thickness, area, or quantity features for quantitatively measuring and analyzing the layer features 156 of the retina (e.g., BM, BMEIS, GCL-IPL, IB-OPR layer, OB-OPR layer, IB-RPE layer, OB-RPE layer, ILM, IPL-INL, IPL-ONL, the ISJ-OSJ layer, OPL-HFL, RNFL-GCL), the fluid features 158 (e.g., IRF, SRF, PED, or SHRM), and hyperreflective entities 159 (e.g., HRM, IHRM, HRF) based on the nine macular subfields determined from the Early Treatment Diabetic Retinopathy Study (ETDRS) grid. In certain embodiments, the one or more volumetric measurements of layer features 156, fluid features 158, and hyperreflective entities 159 may be then analyzed to determine any outliers 152 and longitudinal data dynamics 154.

In certain embodiments, the one or more volumetric measurements of layer features 156, fluid features 158, and hyperreflective entities 159 may be then provided to a machine-learning (ML) functional block 160. In certain embodiments, the ML functional block 160 may be utilized to perform one or more dimensionality reduction, feature selection, and classification tasks based on the one or more volumetric measurements of layer features 156, fluid features 158, and hyperreflective entities 159 and the retinal segmentation, classification, and feature extraction pipeline 104 may generate one or more final outputs.

For example, in accordance with the presently disclosed embodiments, the retinal segmentation, classification, and feature extraction pipeline 104 may generate one or more final outputs of a classification of a patient as having one or more of diabetic retinopathy (DR), diabetic macular edema (DME), age-related macular degeneration (AMD), neovascular age-related macular degeneration (nAMD), geographic atrophy (GA), macular atrophy (MA), and retinal vein occlusion (RVO) based on the one or more volumetric measurements of layer features 156, fluid features 158, and hyperreflective entities 159. In some embodiments, the retinal segmentation, classification, and feature extraction pipeline 104 may further generate one or more final outputs of a classification of one or more treatments to treat a retinal-diseased eye of a patient, determine a risk of progression of retinal disease in the eye of the patient, or to identify an effective treatment regimen to treat a retinal-diseased eye of a patient.

In certain embodiments, the one or more final outputs may be then provided for downstream uses, such as by clinicians 170A (e.g., ophthalmologists, optometrists), biomarker scientists 170B, data scientists 170C, or for data storage and/or sharing 170D. For example, in some embodiments, a report may be generated based on the one or more final outputs of the retinal segmentation, classification, and feature extraction pipeline 104. For example, in one embodiment, the report may include a clinical report that may be associated with one or more retinal patients to be provided and displayed, for example, to clinicians 170A (e.g., ophthalmologists, optometrists) for purposes of research and/or the diagnosis, prognosis, and treatment of the one or more retinal patients. In another embodiment, the report may include an interpretability and/or explainability report that may be associated with the retinal segmentation, classification, and feature extraction pipeline 104 to be provided and displayed, for example, to one or more data scientists 170C for purposes of ascertaining and elucidating the prediction and decision-making behaviors of the retinal segmentation, classification, and feature extraction pipeline 104.

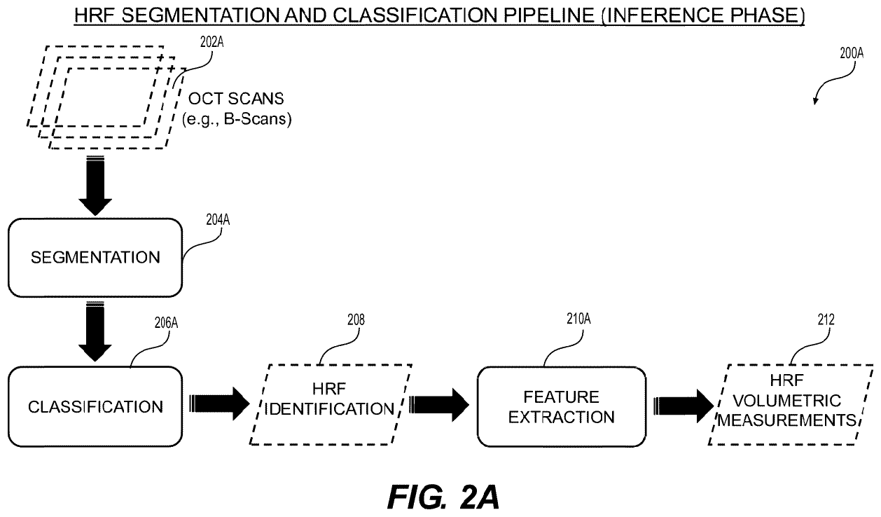

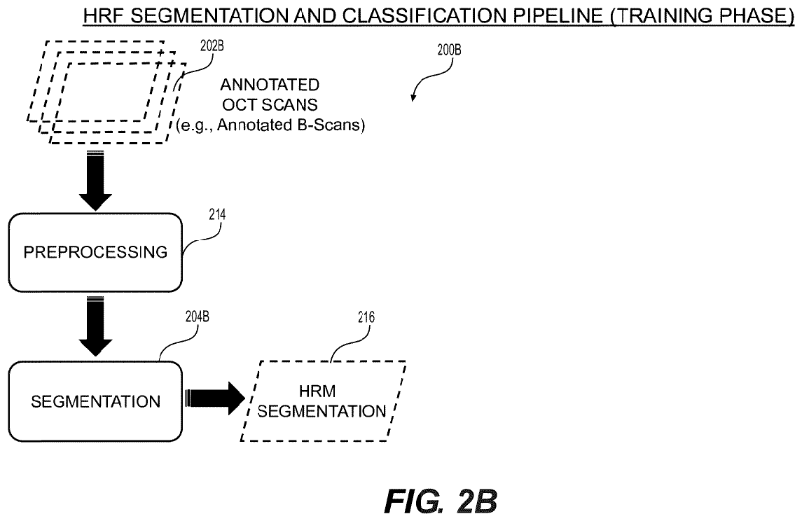

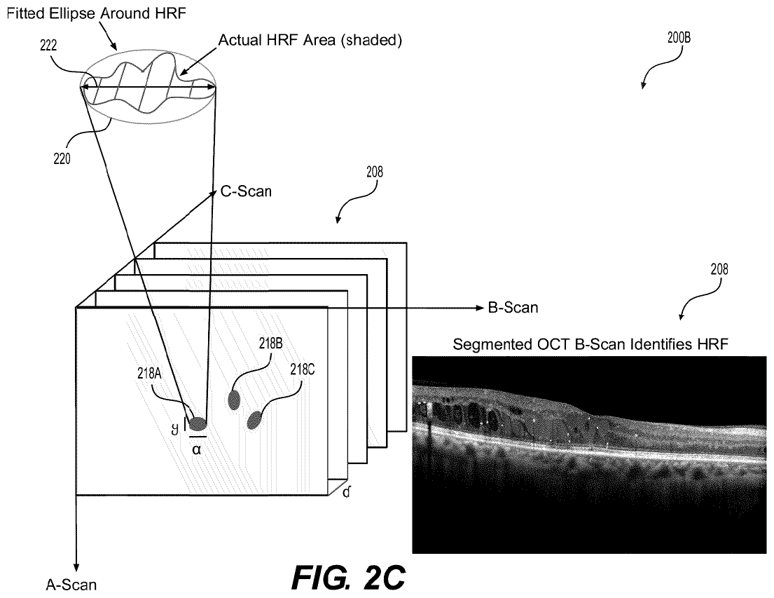

FIG. 2A illustrates a diagram 200A of an inference phase of an HRF segmentation and classification pipeline suitable for detecting and quantifying HRF in the retina of an eye of a patient, in accordance with the presently disclosed embodiments. In one embodiment, the HRF segmentation and classification pipeline may be a subset of the retinal segmentation, classification, and feature extraction pipeline 104 as discussed above with respect to FIG. 1. As depicted by FIG. 2A, in certain embodiments, one or more OCT scans 202A of a retina of an eye of a patient may be accessed and inputted into a segmentation machine-learning model 204A. For example, in some embodiments, the one or more OCT scans 202A may each include, for example, an OCT B-scan of the retina of the eye of the patient. In certain embodiments, the segmentation model 204A may include a semantic segmentation model (e.g., FRRN, FCN, U-Net, PSPNet, FCDenseNet, RefineNet, DeepLabV3, DeepLabV+, SegNet or similar semantic segmentation DCNN) that may be trained to generate one or more predicted segmentation maps identifying a set of hyperreflective entities. For example, in some embodiments, the identified set of hyperreflective entities may include HRM, IHRM, HRF, or other similar HRM that may be associated with one or more retinal diseases.

In certain embodiments, the identified set of hyperreflective entities may be then inputted into a classification module 206A. For example, in some embodiments, the classification module 206A may include an image-processing-based algorithm or similar process that may be utilized to perform diametral measurements of the hyperreflective entities (e.g., HRM, IHRM, HRF) identified by the segmentation model 204A. For example, as will be further illustrated with respect to FIG. 2C below, the classification module 206A may perform the diametral measurements by associating an ellipse with each identified hyperreflective entity (e.g., HRM, IHRM, HRF) and determining a diameter of a longest axis of the ellipses associated with the identified hyperreflective entity (e.g., HRM, IHRM, HRF).

In certain embodiments, the classification module 206A may then estimate a diameter of each identified hyperreflective entity (e.g., HRM, IHRM, HRF) based on the determined diameter of the respective ellipses associated with each identified hyperreflective entity. In certain embodiments, the classification module 206A may then identify (e.g., classify) HRF 208 in the retina of the eye of the patient based on whether the estimated diameter (e.g., estimated based on the determined diameter of the respective ellipses associated with each identified hyperreflective entity) satisfy a diametral threshold. In one embodiment, the diametral threshold may be a diameter or diameter range defined as to distinguish HRF from other hyperreflective entities (e.g., HRM, IHRM, or other HRM) that may be present in the one or more OCT scans 202A and segmented by the segmentation model 204A.

For example, in some embodiments, the classification module 206A may classify a hyperreflective entity as HRF 208 when the estimated diameter of the hyperreflective entity includes a diameter of approximately 50 μm or less. In another embodiment, the classification module 206A may classify a hyperreflective entity as HRF 208 when the estimated diameter of the hyperreflective entity includes a diameter of approximately 40 μm or less, approximately 35 μm or less, approximately 30 μm or less, approximately 25 μm or less, approximately 20 μm or less, approximately 15 μm or less, or approximately 10 μm or less. In other embodiments, the classification module 206A may classify a hyperreflective entity as IHRM when the estimated diameter of the hyperreflective entity includes a diameter within a range of approximately 50 μm to 100 μm. In another embodiment, the classification module 206A may classify a hyperreflective entity as IHRM when the estimated diameter of the hyperreflective entity includes a diameter within a range of approximately 50 μm to 90 μm, approximately 50 μm to 80 μm, approximately 50 μm to 70 μm, or approximately 50 μm to 60 μm.

In certain embodiments, to further refine the hyperreflective entities identified as HRF 208, the predictions of the one or more label maps 144 (e.g., including one or more OCT B-scans with the layers of the retina segmented and annotated) may be utilized to narrow the areas within the segmented OCT B-scans in which hyperreflective entities identified as HRF 208 may be identified. For example, in some embodiments, hyperreflective entities identified as HRF 208 detected outside of one or more particular segmented retinal layer boundaries may be discarded, and only the hyperreflective identities identified as HRF 208 within the one or more particular segmented retinal layer boundaries may be passed to the feature extraction module 210A. For example, in one embodiment, only the hyperreflective entities identified as HRF 208 detected between the ILM and OPL-HFL layers may correspond to identified HRF 208 in the inner retina and only the hyperreflective entities identified as HRF 208 detected between the OPL-HFL and RPE layers may correspond to identified HRF 208 in the outer retina.

In certain embodiments, the identified HRF 208 may be then provided to a feature extraction module 210A. For example, in some embodiments, the feature extraction module 210A may include an image-processing-based algorithm that may be utilized to determine one or more volumetric measurements 212 of the identified HRF 208. For example, in certain embodiments, based on mapping information of the ETDRS grid and the identified HRF 208, the feature extraction module 210A (e.g., image-processing-based algorithm) may calculate one or more volumetric measurements 212, including, for example, a total volume of the identified HRF 208, a volume of the identified HRF 208 with respect to one or more subfields of the ETDRS grid, a total area of the identified HRF 208, an area of the identified HRF 208 with respect to one or more subfields of the ETDRS grid, a thickness of the identified HRF 208 with respect to one or more subfields of the ETDRS grid, a total quantity of the identified HRF 208, a quantity of the identified HRF 208 with respect to one or more subfields of the ETDRS grid, and so forth.

In this way, the presently disclosed embodiments may provide an HRF segmentation and classification pipeline that may be suitable for accurately detecting and quantifying HRF 208 in the retina of an eye of a patient, in which the HRF 208 may be specifically defined as a hyperreflective entity having an estimated diameter 50 μm or less. Indeed, by providing a HRF segmentation and classification pipeline suitable for accurately detecting and quantifying HRF 208 in the retina of an eye of a patient, the present embodiments accurately and efficiently identifies a clinically-significant biomarker of visual acuity and morphological changes in many retinal diseases, such as DR, DME, AMD, nAMD, GA, MA, and RVO. Additionally, the provided HRF segmentation and classification pipeline may be further suitable for utilizing the detected and quantified HRF 208 to predict retinal disease progression and patient treatment response in accordance with the presently disclosed embodiments.

FIG. 2B illustrates a diagram 200B of a training phase of an HRF segmentation and classification pipeline suitable for detecting and quantifying HRF in the retina of an eye of a patient, in accordance with the presently disclosed embodiments. As depicted by FIG. 2B, in certain embodiments, the HRF segmentation and classification pipeline may include a preprocessing module 214 and segmentation model 204B. The segmentation model 204B may correspond to the segmentation model 204A discussed above with respect to FIG. 2A. In some embodiments, the segmentation model 204B may include one or more machine-learning models that may be trained end-to-end to identify HRM.

As depicted by FIG. 2B, in certain embodiments, a data set of sparsely annotated OCT scans 202B of a retina of an eye of one or more patients may be accessed and preprocessed by way of the preprocessing module 214. For example, in some embodiments, the data set of sparsely annotated OCT scans 202B may include a data set of OCT B-scans, in which hyperreflective entities have been sparsely annotated by one or more human graders for training the segmentation model 204B. In one embodiment, the sparse annotations of hyperreflective entities may include a bounding geometry encompassing a number of hyperreflective entities. In certain embodiments, the preprocessing module 214 may then preprocess the data set of sparsely annotated OCT scans 202B by performing an adjustment of the bounding geometry to reduce a size of the bounding geometry so as to annotate only a single hyperreflective entity (e.g., as opposed to the bounding geometry encompassing multiple hyperreflective entities at once).

For example, in some embodiments, the preprocessing module 214 may utilize an Otsu's thresholding algorithm (e.g., dynamic thresholding) or other similar image-processing-based algorithm that may be suitable for binarizing the data set of sparsely annotated OCT scans 202B based on pixel intensities. The bounding geometry may then be reduced to encompass hyperreflective entities having the brightest intensities, which may better represent HRF or IHRM for training the segmentation model 204B. In certain embodiments, the segmentation model 204B may be then trained utilizing the data set of sparsely annotated OCT scans 202B as preprocessed by the preprocessing module 214. For example, in certain embodiments, the preprocessed data set of sparsely annotated OCT scans 202B may be partitioned into a model-training data set and a model-validation data set. In certain embodiments, the model-training data set may be input into the segmentation model 204B for training the segmentation model 204B to identify a set of hyperreflective entities 216 (e.g., HRM, IHRM, HRF). In certain embodiments, once trained, performance of the segmentation model 204B may be then evaluated utilizing the model-validation data set.

FIG. 2C illustrates a diagram 200C of a classification stage of an HRF segmentation and classification pipeline, in accordance with the presently disclosed embodiments. In certain embodiments, as previously discussed above with respect to FIG. 2A, the classification stage may be performed by the classification module 206B. For example, as depicted by FIG. 2C, after the hyperreflective entities 218A, 218B, and 218C (e.g., HRM, IHRM, HRF) are identified by the segmentation model 204A, for example, one or more diametral measurements of the hyperreflective entities 218A, 218B, and 218C (e.g., HRM, IHRM, HRF) may be performed. In certain embodiments, the one or more diametral measurements may be performed by associating an ellipse 220 with each of the identified hyperreflective entities 218A, 218B, and 218C (e.g., HRM, IHRM, HRF) and determining a diameter of a longest axis 222 of the ellipse 220 associated with each of the hyperreflective entities 218A, 218B, and 218C (e.g., HRM, IHRM, HRF). Specifically, in some embodiments, an instance of HRF may be assumed to be an ellipse with a length a as measured along the longest axis 222 of the ellipse 220, for example.

In certain embodiments, HRF in the retina of the eye of the patient may then be identified (e.g., classified) based on whether the one or more diametral measurements satisfy a diametral threshold. In accordance with the presently disclosed embodiments, a hyperreflective entity is identified (e.g., classified) as HRF when the one or more diametral measurements of the hyperreflective entity includes a diameter of 50 μm or less (e.g., α=≤50 μm). In other embodiments, a hyperreflective entity is identified (e.g., classified) as IHRM when the one or more diametral measurements of the hyperreflective entity includes a diameter within a range of approximately 50 μm to 100 μm (e.g., 50 μm<α≤100 μm). In one embodiment, as further depicted by FIG. 2C, volume of an identified instance of HRF may be computed as a product of the actual 2D area and the depth d, in which depth d may be defined as the distance between OCT B-Scans.

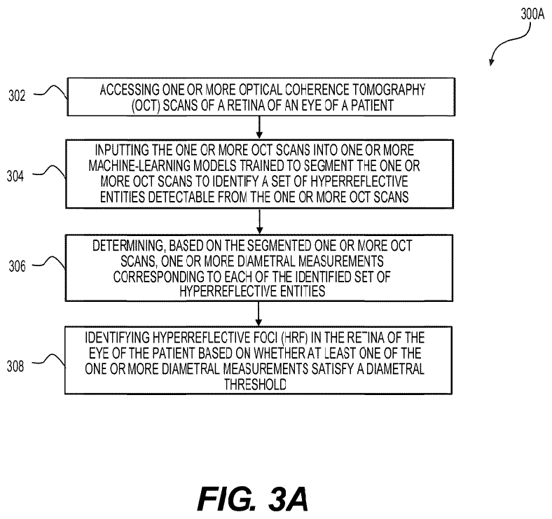

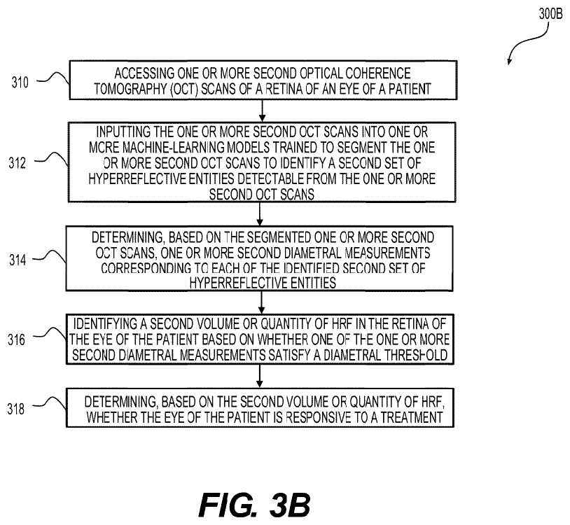

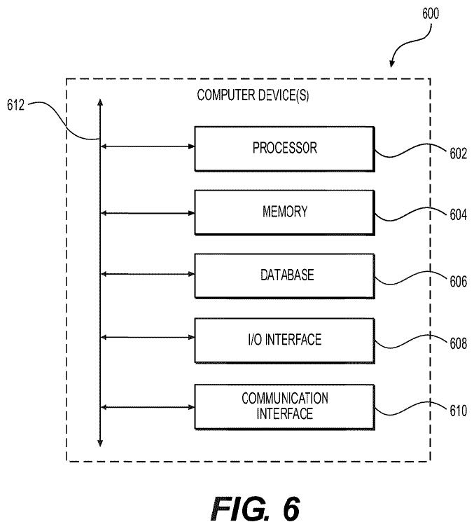

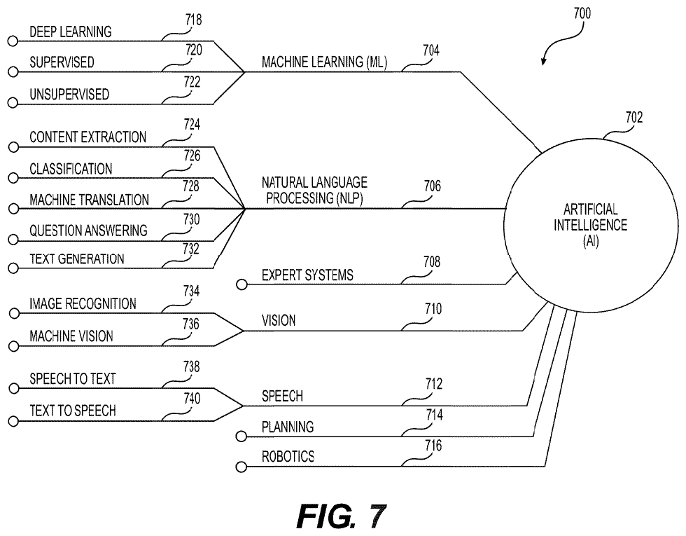

FIG. 3A illustrates a flow diagram of a method 300A for detecting and quantifying HRF in the retina of an eye of a patient, in accordance with the disclosed embodiments. The method 300A may be performed utilizing one or more processing devices (e.g., computing device(s) and artificial intelligence architecture to be discussed below with respect to FIGS. 6 and 7) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other artificial intelligence (AI)/machine-learning (ML) accelerators device(s) that may be suitable for processing medical data and making one or more predictions or decisions based thereon), firmware (e.g., microcode), or some combination thereof.

The method 300A may include at block 302 with one or more processing devices accessing one or more optical coherence tomography (OCT) scans of a retina of an eye of a patient. For example, the one or more processing devices may receive one or more OCT B-scans of a retina of an eye of a patient. The method 300A may include at block 304 the one or more processing devices inputting the one or more OCT scans into one or more machine-learning models trained to segment the one or more OCT scans to identify a set of hyperreflective entities detectable from the one or more OCT scans. For example, in some embodiments, one or more OCT B-scans of a retina of an eye of a patient may be inputted into a semantic segmentation model (e.g., FRRN, FCN, U-Net, PSPNet, FCDenseNet, RefineNet, DeepLabV3, DeepLabV+, SegNet or similar semantic segmentation DCNN), which may generate one or more segmentation maps identifying a set of hyperreflective entities (e.g., HRM, IHRM, HRF).

The method 300A may include at block 306 the one or more processing devices determining, based on the segmented one or more OCT scans, one or more diametral measurements corresponding to each of the identified set of hyperreflective entities. For example, in some embodiments, the one or more processing devices may perform diametral measurements of the hyperreflective entities (e.g., HRM, IHRM, HRF) identified by the semantic segmentation model. For example, the diametral measurements may be performed by associating an ellipse with each identified hyperreflective entity (e.g., HRM, IHRM, HRF) and determining a diameter of a longest axis of the ellipses associated with the identified hyperreflective entity (e.g., HRM, IHRM, HRF).

The method 300A may include at block 308 the one or more processing devices identifying hyperreflective foci (HRF) in the retina of the eye of the patient based on whether at least one of the one or more diametral measurements satisfy a diametral threshold. For example, in some embodiments, the one or more processing devices may classify a hyperreflective entity as HRF when the one or more diametral measurements of the hyperreflective entity includes a minimum diameter of approximately 50 μm. In other embodiments, the one or more processing devices may classify a hyperreflective entity as IHRM when the one or more diametral measurements of the hyperreflective entity includes a diameter within a range of approximately 50 μm to 100 μm. In certain embodiments, the one or more processing devices may discard any hyperreflective entity when the one or more diametral measurements of the hyperreflective entity includes a diameter greater than 100 μm.

FIG. 3B illustrates a flow diagram of a method 300B for determining, based on a change in volume or quantity of HRF, whether an eye of a patient is responsive to a treatment, in accordance with the disclosed embodiments. The method 300B may be performed utilizing one or more processing devices (e.g., computing device(s) and artificial intelligence architecture to be discussed below with respect to FIGS. 6 and 7) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other artificial intelligence (AI)/machine-learning (ML) accelerators device(s) that may be suitable for processing medical data and making one or more predictions or decisions based thereon), firmware (e.g., microcode), or some combination thereof.

The method 300B may include at block 310 one or more processing devices receiving one or more second optical coherence tomography (OCT) scans of a retina of an eye of a patient. For example, the one or more processing devices may receive one or more second OCT B-scans of a retina of an eye of a patient, for example, corresponding to OCT B-scans captured after some period of time in which the patient has undergone one or more treatments and/or treatment regimens. The method 300B may include at block 312 the one or more processing devices inputting the one or more second OCT scans into one or more machine-learning models trained to segment the one or more second OCT scans to identify a second set of hyperreflective entities detectable from the one or more second OCT scans. For example, in some embodiments, one or more second OCT B-scans of the retina of the eye of the patient may be inputted into a semantic segmentation model (e.g., FRRN, FCN, U-Net, PSPNet, FCDenseNet, RefineNet, DeepLabV3, DeepLabV+, SegNet or similar semantic segmentation DCNN), which may generate one or more segmentation maps identifying a set of hyperreflective entities (e.g., HRM, IHRM, HRF).

The method 300B may include at block 314 the one or more processing devices determining, based on the segmented one or more second OCT scans, one or more second diametral measurements corresponding to each of the identified second set of hyperreflective entities. For example, in some embodiments, the one or more processing devices may perform diametral measurements of the hyperreflective entities (e.g., HRM, IHRM, HRF) identified by the semantic segmentation model. For example, the diametral measurements may be performed by associating an ellipse with each identified hyperreflective entity (e.g., HRM, IHRM, HRF) and determining a diameter of a longest axis of the ellipses associated with the identified hyperreflective entity (e.g., HRM, IHRM, HRF).

The method 300B may include at block 316 the one or more processing devices identifying a second volume or quantity of hyperreflective foci (HRF) in the retina of the eye of the patient based on whether at least one of the one or more second diametral measurements satisfy a diametral threshold. For example, in some embodiments, the one or more processing devices may classify a hyperreflective entity as HRF when the one or more diametral measurements of the hyperreflective entity includes a minimum diameter of approximately 50 μm. In other embodiments, the one or more processing devices may classify a hyperreflective entity as IHRM when the one or more diametral measurements of the hyperreflective entity includes a diameter within a range of approximately 50 μm to 100 μm.

The method 300B may include at block 318 the one or more processing devices determining, based on the second volume or quantity of HRF, whether the eye of the patient is responsive to a treatment. For example, in some embodiments, the second volume or quantity of HRF as detected from the one or more second OCT B-scans may be compared to a first volume or quantity of HRF as detected from one or more first OCT B-scans of the retina of the eye of the patient captured prior to the patient having undergone the one or more treatments and/or treatment regimens. In certain embodiments, a reduction in the volume or quantity of HRF may indicate that the one or more treatments and/or treatment regimens are effective.

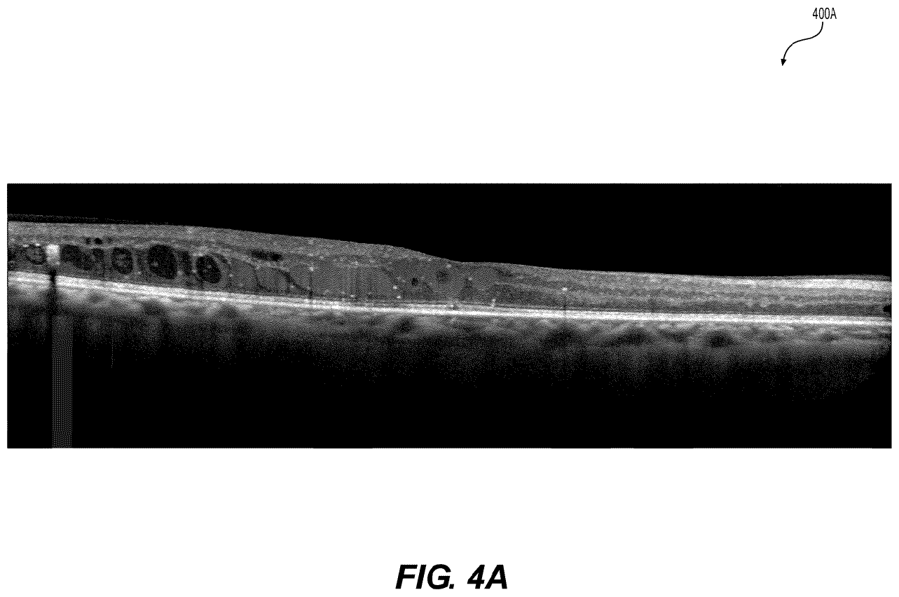

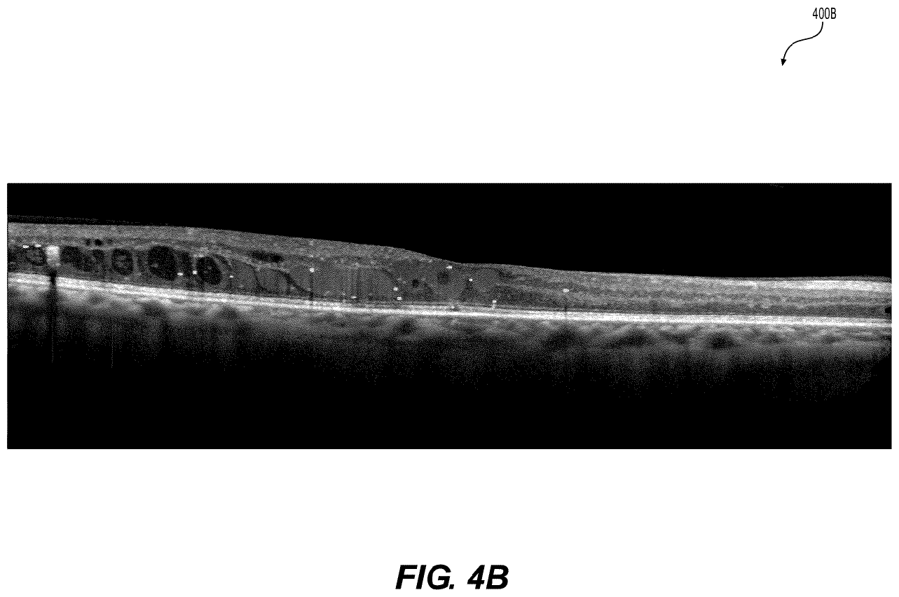

FIG. 4A illustrates an enlarged example image of an OCT B-scan 400A, in accordance with the presently disclosed embodiments. In certain embodiments, the OCT B-scan 400A may be an OCT B-scan of the retina of an eye of a patient, in which the retina of the eye of the patient includes HRF as undetected and unquantified in accordance with the presently disclosed embodiments.

FIG. 4B illustrates an enlarged example image of a segmented and classified OCT B-scan 400B, in accordance with the presently disclosed embodiments. In certain embodiments, the segmented OCT B-scan 400B may be a segmented and classified OCT B-scan of the retina of an eye of a patient, in which the segmented and classified OCT B-scan 400B illustrates a result of a segmentation and classification of the OCT B-scan 400A as discussed above with respect to FIG. 4A. As depicted, the segmented and classified OCT B-scan 400B identifies HRF (e.g., as displayed by the yellow-colored objects that appear to be rectangular and/or square), in accordance with the presently disclosed embodiments.

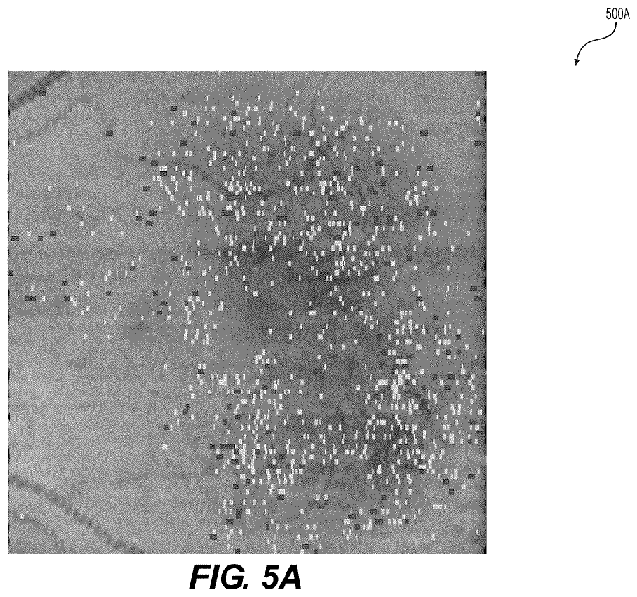

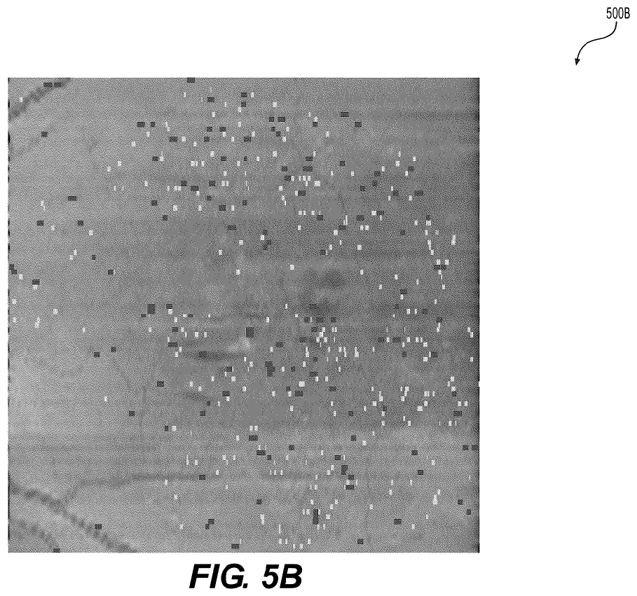

FIGS. 5A and 5B illustrate enlarged example images of an en face image 500A and en face image 500B, respectively, in accordance with the presently disclosed embodiments. In certain embodiments, the en face image 500A and the en face image 500B may each include a rendering of an en face image generated following the segmentation, classification, and feature extraction of one or OCT B-scans as generally described herein. As depicted, the en face image 500A may include a greater volume and quantity of HRF (e.g., as displayed by the yellow-colored or lighter objects) and IHRM (e.g., as displayed by the blue-colored or darker objects) as compared to the volume and quantity of HRF (e.g., as displayed by the yellow-colored objects or lighter objects) and IHRM (e.g., as displayed by the blue-colored objects or darker objects) depicted in the en face image 500B. In one embodiment, the en face image 500A may include an example en face image of an eye of a patient prior to having undergone any treatment and/or treatment regimen, while the en face image 500B may include an example en face image of the eye of the patient after having undergone a treatment and/or treatment regimen.

In one embodiment, the treatment may include an anti-vascular endothelial growth factor (anti-VEGF) antibody, an anti-vascular endothelial growth factor-A (anti-VEGF-A) antibody, an anti-anangiopoietin-2 (anti-Ang-2) antibody, or some combination thereof. For example, in one embodiment, the anti-VEGF-A antibody may include faricimab-svoa and the anti-Ang-2 antibody may include faricimab-svoa. In another embodiment, the anti-VEGF antibody may be selected from the group including ranibizumab, aflibercept, brolucizumab, bevacizumab, and pegaptanib sodium.

FIG. 6 illustrates an example of one or more computing device(s) 600 that may be utilized for detecting and quantifying HRF in the retina of an eye of a patient, in accordance with the disclosed embodiments. In certain embodiments, the one or more computing device(s) 600 may perform one or more steps of one or more methods described or illustrated herein. In certain embodiments, the one or more computing device(s) 600 provide functionality described or illustrated herein. In certain embodiments, software running on the one or more computing device(s) 600 performs one or more steps of one or more methods described or illustrated herein, or provides functionality described or illustrated herein. Certain embodiments include one or more portions of the one or more computing device(s) 600.

This disclosure contemplates any suitable number of computing systems 600. This disclosure contemplates one or more computing device(s) 600 taking any suitable physical form. As example and not by way of limitation, one or more computing device(s) 600 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the one or more computing device(s) 600 may be unitary or distributed; span multiple locations; span multiple machines; span multiple data centers; or reside in a cloud, which may include one or more cloud components in one or more networks.

Where appropriate, the one or more computing device(s) 600 may perform without substantial spatial or temporal limitation one or more steps of one or more methods described or illustrated herein. As an example, and not by way of limitation, the one or more computing device(s) 600 may perform, in real-time or in batch mode, one or more steps of one or more methods described or illustrated herein. The one or more computing device(s) 600 may perform, at different times or at different locations, one or more steps of one or more methods described or illustrated herein, where appropriate.

In certain embodiments, the one or more computing device(s) 600 includes a processor 602, memory 604, database 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement. In certain embodiments, processor 602 includes hardware for executing instructions, such as those making up a computer program. As an example, and not by way of limitation, to execute instructions, processor 602 may retrieve (or fetch) the instructions from an internal register, an internal cache, memory 604, or database 606; decode and execute them; and then write one or more results to an internal register, an internal cache, memory 604, or database 606. In certain embodiments, processor 602 may include one or more internal caches for data, instructions, or addresses. This disclosure contemplates processor 602 including any suitable number of any suitable internal caches, where appropriate. As an example, and not by way of limitation, processor 602 may include one or more instruction caches, one or more data caches, and one or more translation lookaside buffers (TLBs). Instructions in the instruction caches may be copies of instructions in memory 604 or database 606, and the instruction caches may speed up retrieval of those instructions by processor 602.

Data in the data caches may be copies of data in memory 604 or database 606 for instructions executing at processor 602 to operate on; the results of previous instructions executed at processor 602 for access by subsequent instructions executing at processor 602 or for writing to memory 604 or database 606; or other suitable data. The data caches may speed up read or write operations by processor 602. The TLBs may speed up virtual-address translation for processor 602. In certain embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In certain embodiments, memory 604 includes main memory for storing instructions for processor 602 to execute or data for processor 602 to operate on. As an example, and not by way of limitation, the one or more computing device(s) 600 may load instructions from database 606 or another source (such as, for example, another one or more computing device(s) 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604.

In certain embodiments, processor 602 executes only instructions in one or more internal registers, internal caches, or memory 604 (as opposed to database 606 or elsewhere) and operates only on data in one or more internal registers, internal caches, or memory 604 (as opposed to database 606 or elsewhere). One or more memory buses (which may each include an address bus and a data bus) may couple processor 602 to memory 604. Bus 612 may include one or more memory buses, as described below. In certain embodiments, one or more memory management units (MMUs) reside between processor 602 and memory 604 and facilitate accesses to memory 604 requested by processor 602. In certain embodiments, memory 604 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memory devices 604, where appropriate. Although this disclosure describes and illustrates particular memory, this disclosure contemplates any suitable memory.

In certain embodiments, database 606 includes mass storage for data or instructions. As an example, and not by way of limitation, database 606 may include a hard disk drive (HDD), a floppy disk drive, flash memory, an optical disc, a magneto-optical disc, magnetic tape, or a Universal Serial Bus (USB) drive, or a combination of two or more of these. Database 606 may include removable or non-removable (or fixed) media, where appropriate. Database 606 may be internal or external to the one or more computing device(s) 600, where appropriate. In certain embodiments, database 606 is non-volatile, solid-state memory. In certain embodiments, database 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), flash memory, or a combination of two or more of these. This disclosure contemplates mass database 606 taking any suitable physical form. Database 606 may include one or more storage control units facilitating communication between processor 602 and database 606, where appropriate. Where appropriate, database 606 may include one or more databases 606. Although this disclosure describes and illustrates particular storage, this disclosure contemplates any suitable storage.

In certain embodiments, I/O interface 608 includes hardware, software, or both, providing one or more interfaces for communication between the one or more computing device(s) 600 and one or more I/O devices. The one or more computing device(s) 600 may include one or more of these I/O devices, where appropriate. One or more of these I/O devices may enable communication between a person and the one or more computing device(s) 600. As an example, and not by way of limitation, an I/O device may include a keyboard, keypad, microphone, monitor, mouse, printer, scanner, speaker, still camera, stylus, tablet, touch screen, trackball, video camera, another suitable I/O device, or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In certain embodiments, communication interface 610 includes hardware, software, or both providing one or more interfaces for communication (such as, for example, packet-based communication) between the one or more computing device(s) 600 and one or more other computing device(s) 600 or one or more networks. As an example, and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it.

As an example, and not by way of limitation, the one or more computing device(s) 600 may communicate with an ad hoc network, a personal area network (PAN), a local area network (LAN), a wide area network (WAN), a metropolitan area network (MAN), one or more portions of the Internet, or a combination of two or more of these. One or more portions of one or more of these networks may be wired or wireless. As an example, the one or more computing device(s) 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), other suitable wireless network, or a combination of two or more of these. The one or more computing device(s) 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In certain embodiments, bus 612 includes hardware, software, or both coupling components of the one or more computing device(s) 600 to each other. As an example, and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, another suitable bus, or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

FIG. 7 illustrates a diagram 700 of an example artificial intelligence (AI) architecture 702 (which may be included as part of the one or more computing device(s) 600 as discussed above with respect to FIG. 6) that may be utilized for detecting and quantifying HRF in the retina of an eye of a patient, in accordance with the presently disclosed embodiments. In certain embodiments, the AI architecture 702 may be implemented utilizing, for example, one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), and/or other artificial intelligence (AI)/machine-learning (ML) accelerator device(s) that may be suitable for processing various data and making one or more predictions or decisions based thereon), software (e.g., instructions running/executing on one or more processing devices), firmware (e.g., microcode), or some combination thereof.

In certain embodiments, as depicted by FIG. 7, the AI architecture 702 may include machine learning (ML) models 704, natural language processing (NLP) models 706, expert systems 708, computer-based vision models 710, speech recognition models and functions 712, planning models 714, and robotics models and functions 716. In certain embodiments, the ML models 704 may include any statistics-based models that may be suitable for finding patterns across large amounts of data (e.g., “Big Data” such as genomics data, proteomics data, metabolomics data, metagenomics data, transcriptomics data, or other omics data). For example, in certain embodiments, the ML models 704 may include deep learning models 718, supervised learning models 720, and unsupervised learning models 722.

In certain embodiments, the deep learning models 718 may include any artificial neural networks (ANNs) that may be utilized to learn deep levels of representations and abstractions from large amounts of data. For example, the deep learning models 718 may include ANNs, such as a perceptron, a multilayer perceptron (MLP), an autoencoder (AE), a convolution neural network (CNN), a recurrent neural network (RNN), long short term memory (LSTM), a gated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, and so forth.

In certain embodiments, the supervised learning models 720 may include any algorithms that may be utilized to apply, for example, what has been learned in the past to new data using labeled examples for predicting future events. For example, starting from the analysis of a known training data set, the supervised learning models 720 may produce an inferred function to make predictions about the output values. The supervised learning models 720 may also compare its output with the correct and intended output and find errors in order to modify the supervised learning models 720 accordingly. On the other hand, the unsupervised learning models 722 may include any algorithms that may be applied, for example, when the data used to train the unsupervised learning models 722 are neither classified nor labeled. For example, the unsupervised learning models 722 may study and analyze how systems may infer a function to describe a hidden structure from unlabeled data.

In certain embodiments, the NLP models 706 may include any algorithms or functions that may be suitable for automatically manipulating natural language, such as speech and/or text. For example, in some embodiments, the NLP models 706 may include content extraction models 724, classification models 726, machine translation models 728, question answering (QA) models 730, and text generation models 732. In certain embodiments, the content extraction models 724 may include a means for extracting text or images from electronic documents (e.g., webpages, text editor documents, and so forth) to be utilized, for example, in other applications.

In certain embodiments, the classification models 726 may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon. The machine translation models 728 may include any algorithms or functions that may be suitable for automatically converting source text in one language, for example, into text in another language. The QA models 730 may include any algorithms or functions that may be suitable for automatically answering questions posed by humans in, for example, a natural language, such as that performed by voice-controlled personal assistant devices. The text generation models 732 may include any algorithms or functions that may be suitable for automatically generating natural language texts.

In certain embodiments, the expert systems 708 may include any algorithms or functions that may be suitable for simulating the judgment and behavior of a human or an organization that has expert knowledge and experience in a particular field (e.g., stock trading, medicine, sports statistics, and so forth). The computer-based vision models 710 may include any algorithms or functions that may be suitable for automatically extracting information from images (e.g., photo images, video images). For example, the computer-based vision models 710 may include image recognition algorithms 734 and machine vision algorithms 736. The image recognition algorithms 734 may include any algorithms that may be suitable for automatically identifying and/or classifying objects, places, people, and so forth that may be included in, for example, one or more image frames or other displayed data. The machine vision algorithms 736 may include any algorithms that may be suitable for allowing computers to “see”, or, for example, to rely on image sensors cameras with specialized optics to acquire images for processing, analyzing, and/or measuring various data characteristics for decision making purposes.

In certain embodiments, the speech recognition models 712 may include any algorithms or functions that may be suitable for recognizing and translating spoken language into text, such as through automatic speech recognition (ASR), computer speech recognition, speech-to-text (STT) 738, or text-to-speech (TTS) 740 in order for the computing to communicate via speech with one or more users, for example. In certain embodiments, the planning models 714 may include any algorithms or functions that may be suitable for generating a sequence of actions, in which each action may include its own set of preconditions to be satisfied before performing the action. Examples of AI planning may include classical planning, reduction to other problems, temporal planning, probabilistic planning, preference-based planning, conditional planning, and so forth. Lastly, the robotics models 716 may include any algorithms, functions, or systems that may enable one or more devices to replicate human behavior through, for example, motions, gestures, performance tasks, decision-making, emotions, and so forth.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g. method, may be claimed in another claim category, e.g. system, as well. The dependencies or references back in the attached claims are chosen for formal reasons only. However, any subject matter resulting from a deliberate reference back to any previous claims (in particular multiple dependencies) may be claimed as well, so that any combination of claims and the features thereof are disclosed and may be claimed regardless of the dependencies chosen in the attached claims. The subject-matter which may be claimed comprises not only the combinations of features as set out in the attached claims but also any other combination of features in the claims, wherein each feature mentioned in the claims may be combined with any other feature or combination of other features in the claims. Furthermore, any of the embodiments and features described or depicted herein may be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

### Embodiments

Among the provided embodiments are:
1. A method for identifying hyperreflective foci (HRF) in an eye of a patient, comprising, by one or more computing devices:
   accessing one or more optical coherence tomography (OCT) scans of a retina of the eye of the patient;
   inputting the one or more OCT scans into one or more machine-learning models trained to segment the one or more OCT scans to identify a set of hyperreflective entities detectable from the one or more OCT scans;
   determining, based on the segmented one or more OCT scans, one or more diametral measurements corresponding to each of the identified set of hyperreflective entities; and
   identifying hyperreflective foci (HRF) in the retina of the eye of the patient based on whether at least one of the one or more diametral measurements satisfy a diametral threshold.
2. The method of Embodiment 1, wherein the set of hyperreflective entities comprise a set of a hyperreflective material (HRM), intraretinal hyperreflective material (IHRM), and HRF.
3. The method of any of Embodiments 1-2, wherein identifying HRF in the retina of the eye of the patient comprises identifying a subset of the identified set of hyperreflective entities.
4. The method of any of Embodiments 1-3, further comprising identifying intraretinal hyperreflective material (IHRM) in the retina of the eye of the patient based on whether the at least one of the one or more diametral measurements satisfy a second diametral threshold.
5. The method of any of Embodiments 1-4, wherein the diametral threshold comprises a minimum diameter of approximately 50 microns (μm).
6. The method of any of Embodiments 4-5, wherein the second diametral threshold comprises a diameter range of approximately 50 microns (μm) to 100 μm.
7. The method of Embodiment 1, wherein determining whether the at least one of the one or more diametral measurements satisfy the diametral threshold further comprises:
   for each of the identified set of hyperreflective entities:
      associating an ellipse with the identified hyperreflective entity;
      determining a diameter of a longest axis of the ellipse; and
      estimating, based on the diameter of the longest axis of the ellipse, the at least one of the one or more diametral measurements.
8. The method of Embodiment 1, wherein identifying HRF in the retina of the eye of the patient further comprises classifying the eye of the patient as having at least one of diabetic retinopathy (DR), diabetic macular edema (DME), age-related macular degeneration (AMD), neovascular age-related macular degeneration (nAMD), geographic atrophy (GA), macular atrophy (MA), or retinal vein occlusion (RVO).
9. The method of Embodiment 1, wherein identifying HRF in the retina of the eye of the patient further comprises:
   accessing Early Treatment for Diabetic Retinopathy Study (ETDRS) grid mapping information identifying one or more subfields of the ETDRS grid; and
   determining, based at least in part on the ETDRS grid mapping information, one or more volumetric measurements of the identified HRF.
10. The method of Embodiment 9, wherein the one or more volumetric measurements comprises one or more of a volume of HRF in the retina of the eye of the patient, an area of HRF in the retina of the eye of the patient, or a thickness of HRF in the retina of the eye of the patient.
11. The method of any of Embodiments 9-10, further comprising determining, based at least in part on the ETDRS grid mapping information, a volume of HRF in the retina of the eye of the patient with respect to at least one of the identified one or more subfields.
12. The method of Embodiment 11, wherein determining the volume of HRF in the retina of the eye of the patient comprises determining a reduction in the volume of HRF in the retina of the eye of the patient.
13. The method of Embodiment 11, wherein the at least one of the identified one or more subfields comprises an outer retina subfield of the ETDRS grid.
14. The method of any of Embodiments 9-13, further comprising determining, based at least in part on the ETDRS grid mapping information, a quantity of HRF in the retina of the eye of the patient with respect to at least one of the identified one or more subfields.
15. The method of any of Embodiments 9-14, further comprising:
   accessing an en face image of the retina of the eye of the patient, wherein the en face image is associated with the one or more OCT scans; and
   mapping, based at least in part on the ETDRS grid mapping information, the identified HRF to the en face image.
16. The method of Embodiment 1, wherein the one or more machine-learning models comprise at least one semantic segmentation model.
17. The method of Embodiment 16, wherein the at least one semantic segmentation model comprises a U-Net architecture.
18. The method of Embodiment 1, further comprising training the one or more machine-learning models by:
   accessing a data set of OCT scans of a retina of an eye of one or more patients, wherein the data set of OCT scans comprise sparse annotations of HRF in the retina of the eye of the one or more patients;
   partitioning the data set of OCT scans into a model-training data set and a model-validation data set;
   training, based on the model-training data set, the one or more machine-learning models to segment OCT scans to identify sets of hyperreflective entities detectable from the OCT scans, wherein the identified sets of hyperreflective entities comprise hyperreflective material (HRM); and
   evaluating the one or more machine-learning models based on the model-validation data set.
19. The method of Embodiment 18, further comprising identifying HRF in the retina of the eye of the one or more patients based on whether one or more diametral measurements of the HRM satisfy a predetermined diametral threshold.
20. The method of Embodiment 18, wherein the sparse annotations of HRF comprise a bounding geometry encompassing a plurality of instances of HRF, the method further comprising:
   prior to training the one or more machine-learning models, performing an adjustment of the bounding geometry by reducing a size of the bounding geometry, the size of the bounding geometry being reduced to annotate a single instance of HRF.
21. The method of Embodiment 1, wherein the one or more OCT scans comprise one or more first OCT scans of the retina of the eye of the patient being captured at an initial date, and wherein the identified HRF comprises a first volume of HRF, the method further comprising:
   accessing one or more second OCT scans of the retina of the eye of the patient;
   inputting the one or more second OCT scans into the one or more machine-learning models to segment the one or more second OCT scans to identify a second set of hyperreflective entities detectable from the one or more second OCT scans;
   determining, based on the segmented one or more second OCT scans, one or more second diametral measurements corresponding to each of the identified second set of hyperreflective entities;
   identifying a second volume of HRF in the retina of the eye of the patient based on whether at least one of the one or more second diametral measurements satisfy the diametral threshold; and
   determining, based on the second volume of HRF, whether the eye of the patient is responsive to a treatment.
22. The method of Embodiment 21, further comprising determining, based on the second volume of HRF, a degree to which the eye of the patient is responsive to the treatment.
23. The method of any of Embodiments 21-22, wherein the eye of the patient is responsive to the treatment when the second volume of HRF is less than the first volume of HRF.
24. The method of any of Embodiments 21-23, wherein the treatment comprises an anti-vascular endothelial growth factor (anti-VEGF) antibody, an anti-vascular endothelial growth factor-A (anti-VEGF-A) antibody, an anti-anangiopoietin-2 (anti-Ang-2) antibody, or a combination thereof.
25. The method of Embodiments 21-24, wherein the anti-VEGF-A antibody comprises faricimab-svoa.
26. The method of Embodiments 21-25, wherein the anti-Ang-2 antibody comprises faricimab-svoa.
27. The method of Embodiments 21-26, wherein the anti-VEGF antibody is selected from the group comprising ranibizumab, aflibercept, brolucizumab, bevacizumab, and pegaptanib sodium.
28. The method of Embodiment 1, further comprising identifying an effective treatment regimen of an anti-vascular endothelial growth factor (anti-VEGF) antibody, an anti-anangiopoietin-2 (anti-Ang-2) antibody, or a combination thereof, to treat the eye of the patient based on a volume or a quantity of the identified HRF.
29. The method of Embodiment 28, wherein identifying the effective treatment regimen comprises identifying, based on the volume or the quantity of the identified HRF, a dosage for administering the anti-VEGF antibody, the anti-Ang-2 antibody, or the combination thereof.
30. The method of any of Embodiments 28-29, wherein identifying the effective treatment regimen comprises identifying, based on the volume or the quantity of the identified HRF, a schedule for administering the anti-VEGF antibody, the anti-Ang-2 antibody, or the combination thereof.
31. The method of any of Embodiments 28-30, wherein identifying the effective treatment regimen comprises identifying, based on the volume or the quantity of the identified HRF, a duration for administering the anti-VEGF antibody, the anti-Ang-2 antibody, or the combination thereof.
32. The method of Embodiment 21, wherein the one or more second OCT scans are captured at one or more dates selected from the group comprising approximately 0.25 months, 0.5 months, 0.75 months, 1 month, 3 months, 6 months, 9 months, 12 months, 15 months, 18 months, 21 months, 24 months, 27 months, 30 months, 33 months, or 36 months from the initial date.
33. A system including one or more computing devices for identifying hyperreflective foci (HRF) in an eye of a patient, the one or more computing devices comprising:
   one or more non-transitory computer-readable storage media including instructions; and
   one or more processors coupled to the one or more storage media, the one or more processors configured to execute the instructions to:
      access one or more optical coherence tomography (OCT) scans of a retina of the eye of the patient;
      input the one or more OCT scans into one or more machine-learning models trained to segment the one or more OCT scans to identify a set of hyperreflective entities detectable from the one or more OCT scans;
      determine, based on the segmented one or more OCT scans, one or more diametral measurements corresponding to each of the identified set of hyperreflective entities; and
      identify hyperreflective foci (HRF) in the retina of the eye of the patient based on whether at least one of the one or more diametral measurements satisfy a diametral threshold.
34. The system of Embodiment 33, wherein the set of hyperreflective entities comprise a set of a hyperreflective material (HRM), intraretinal hyperreflective material (IHRM), and HRF.
35. The system of any of Embodiments 33-34, wherein the instructions to identify HRF in the retina of the eye of the patient further comprise instructions to identifying a subset of the identified set of hyperreflective entities.
36. The system of any of Embodiments 33-35, wherein the instructions further comprise instructions to identify intraretinal hyperreflective material (IHRM) in the retina of the eye of the patient based on whether the at least one of the one or more diametral measurements satisfy a second diametral threshold.
37. The system of any of Embodiments 33-36, wherein the diametral threshold comprises a minimum diameter of approximately 50 microns (μm).
38. The system of any of Embodiments 36-37, wherein the second diametral threshold comprises a diameter range of approximately 50 microns (μm) to 100 μm.
39. The system of Embodiment 33, wherein the instructions to determine whether the at least one of the one or more diametral measurements satisfy the diametral threshold further comprise instructions to:
   for each of the identified set of hyperreflective entities:
      associate an ellipse with the identified hyperreflective entity;
      determine a diameter of a longest axis of the ellipse; and
      estimate, based on the diameter of the longest axis of the ellipse, the at least one of the one or more diametral measurements.
40. The system of Embodiment 33, wherein the instructions to identify HRF in the retina of the eye of the patient further comprise instructions to classify the eye of the patient as having at least one of diabetic retinopathy (DR), diabetic macular edema (DME), age-related macular degeneration (AMD), neovascular age-related macular degeneration (nAMD), geographic atrophy (GA), macular atrophy (MA), or retinal vein occlusion (RVO).
41. The system of Embodiment 33, wherein the instructions to identify HRF in the retina of the eye of the patient further comprise instructions to:
   access Early Treatment for Diabetic Retinopathy Study (ETDRS) grid mapping information identifying one or more subfields of the ETDRS grid; and
   determine, based at least in part on the ETDRS grid mapping information, one or more volumetric measurements of the identified HRF.
42. The system of Embodiment 41, wherein the one or more volumetric measurements comprises one or more of a volume of HRF in the retina of the eye of the patient, an area of HRF in the retina of the eye of the patient, or a thickness of HRF in the retina of the eye of the patient.
43. The system of any of Embodiments 41-42, wherein the instructions further comprise instructions to determine, based at least in part on the ETDRS grid mapping information, a volume of HRF in the retina of the eye of the patient with respect to at least one of the identified one or more subfields.
44. The system of Embodiment 43, wherein the instructions to determine the volume of HRF in the retina of the eye of the patient further comprise instructions to determine a reduction in the volume of HRF in the retina of the eye of the patient.
45. The system of Embodiment 43, wherein the at least one of the identified one or more subfields comprises an outer retina subfield of the ETDRS grid.
46. The system of any of Embodiments 41-45, wherein the instructions further comprise instructions to determine, based at least in part on the ETDRS grid mapping information, a quantity of HRF in the retina of the eye of the patient with respect to at least one of the identified one or more subfields.
47. The system of any of Embodiments 41-46, wherein the instructions further comprise instructions to:
   access an en face image of the retina of the eye of the patient, wherein the en face image is associated with the one or more OCT scans; and
   map, based at least in part on the ETDRS grid mapping information, the identified HRF to the en face image.
48. The system of Embodiment 33, wherein the one or more machine-learning models comprise at least one semantic segmentation model.
49. The system of Embodiment 48, wherein the at least one semantic segmentation model comprises a U-Net architecture.
50. The system of Embodiment 33, wherein the instructions further comprise instructions to train the one or more machine-learning models by:
   accessing a data set of OCT scans of a retina of an eye of one or more patients, wherein the data set of OCT scans comprise sparse annotations of HRF in the retina of the eye of the one or more patients;
   partitioning the data set of OCT scans into a model-training data set and a model-validation data set;
   training, based on the model-training data set, the one or more machine-learning models to segment OCT scans to identify sets of hyperreflective entities detectable from the OCT scans, wherein the identified sets of hyperreflective entities comprise hyperreflective material (HRM); and
   evaluating the one or more machine-learning models based on the model-validation data set.
51. The system of Embodiment 50, wherein the instructions further comprise instructions to identify HRF in the retina of the eye of the one or more patients based on whether one or more diametral measurements of the HRM satisfy a predetermined diametral threshold.
52. The system of Embodiment 50, wherein the sparse annotations of HRF comprise a bounding geometry encompassing a plurality of instances of HRF, and wherein the instructions further comprise instructions to:
   prior to training the one or more machine-learning models, perform an adjustment of the bounding geometry by reducing a size of the bounding geometry, the size of the bounding geometry being reduced to annotate a single instance of HRF.
53. The system of Embodiment 33, wherein the one or more OCT scans comprise one or more first OCT scans of the retina of the eye of the patient being captured at an initial date, wherein the identified HRF comprises a first volume of HRF, and wherein the instructions further comprise instructions to:
   access one or more second OCT scans of the retina of the eye of the patient;
   input the one or more second OCT scans into the one or more machine-learning models to segment the one or more second OCT scans to identify a second set of hyperreflective entities detectable from the one or more second OCT scans;
   determine, based on the segmented one or more second OCT scans, one or more second diametral measurements corresponding to each of the identified second set of hyperreflective entities;
   identify a second volume of HRF in the retina of the eye of the patient based on whether at least one of the one or more second diametral measurements satisfy the diametral threshold; and
   determine, based on the second volume of HRF, whether the eye of the patient is responsive to a treatment.
54. The system of Embodiment 53, wherein the instructions further comprise instructions to determine, based on the second volume of HRF, a degree to which the eye of the patient is responsive to the treatment.
55. The system of any of Embodiments 53-54, wherein the eye of the patient is responsive to the treatment when the second volume of HRF is less than the first volume of HRF.
56. The system of any of Embodiments 53-55, wherein the treatment comprises an anti-vascular endothelial growth factor (anti-VEGF) antibody, an anti-vascular endothelial growth factor-A (anti-VEGF-A) antibody, an anti-anangiopoietin-2 (anti-Ang-2) antibody, or a combination thereof.
57. The system of Embodiments 53-56, wherein the anti-VEGF-A antibody comprises faricimab-svoa.
58. The system of Embodiments 53-57, wherein the anti-Ang-2 antibody comprises faricimab-svoa.
59. The system of Embodiments 53-58, wherein the anti-VEGF antibody is selected from the group comprising ranibizumab, aflibercept, brolucizumab, bevacizumab, and pegaptanib sodium.
60. The system of Embodiment 33, wherein the instructions further comprise instructions to identify an effective treatment regimen of an anti-vascular endothelial growth factor (anti-VEGF) antibody, an anti-anangiopoietin-2 (anti-Ang-2) antibody, or a combination thereof, to treat the eye of the patient based on a volume or a quantity of the identified HRF.
61. The system of Embodiment 60, wherein the instructions to identify the effective treatment regimen further comprise instructions to identify, based on the volume or the quantity of the identified HRF, a dosage for administering the anti-VEGF antibody, the anti-Ang-2 antibody, or the combination thereof.
62. The system of any of Embodiments 60-61, wherein the instructions to identify the effective treatment regimen further comprise instructions to identify, based on the volume or the quantity of the identified HRF, a schedule for administering the anti-VEGF antibody, the anti-Ang-2 antibody, or the combination thereof.
63. The system of any of Embodiments 60-62, wherein the instructions to identify the effective treatment regimen further comprise instructions to identify, based on the volume or the quantity of the identified HRF, a duration for administering the anti-VEGF antibody, the anti-Ang-2 antibody, or the combination thereof.
64. The system of Embodiment 53, wherein the one or more second OCT scans are captured at one or more dates selected from the group comprising approximately 0.25 months, 0.5 months, 0.75 months, 1 month, 3 months, 6 months, 9 months, 12 months, 15 months, 18 months, 21 months, 24 months, 27 months, 30 months, 33 months, or 36 months from the initial date.
65. A non-transitory computer-readable medium comprising instructions that, when executed by one or more processors of one or more computing devices, cause the one or more processors to:
   access one or more optical coherence tomography (OCT) scans of a retina of the eye of the patient;
   input the one or more OCT scans into one or more machine-learning models trained to segment the one or more OCT scans to identify a set of hyperreflective entities detectable from the one or more OCT scans;
   determine, based on the segmented one or more OCT scans, one or more diametral measurements corresponding to each of the identified set of hyperreflective entities; and
   identify hyperreflective foci (HRF) in the retina of the eye of the patient based on whether at least one of the one or more diametral measurements satisfy a diametral threshold.
66. The non-transitory computer-readable medium of Embodiment 65, wherein the set of hyperreflective entities comprise a set of a hyperreflective material (HRM), intraretinal hyperreflective material (IHRM), and HRF.
67. The non-transitory computer-readable medium of any of Embodiments 65-66, wherein the instructions to identify HRF in the retina of the eye of the patient further comprise instructions to identifying a subset of the identified set of hyperreflective entities.
68. The non-transitory computer-readable medium of any of Embodiments 65-67, wherein the instructions further comprise instructions to identify intraretinal hyperreflective material (IHRM) in the retina of the eye of the patient based on whether the at least one of the one or more diametral measurements satisfy a second diametral threshold.
69. The non-transitory computer-readable medium of any of Embodiments 65-68, wherein the diametral threshold comprises a minimum diameter of approximately 50 microns (μm).
70. The non-transitory computer-readable medium of any of Embodiments 68-69, wherein the second diametral threshold comprises a diameter range of approximately 50 microns (μm) to 100 μm.
71. The non-transitory computer-readable medium of Embodiment 65, wherein the instructions to determine whether the at least one of the one or more diametral measurements satisfy the diametral threshold further comprise instructions to:
   for each of the identified set of hyperreflective entities:
      associate an ellipse with the identified hyperreflective entity;
      determine a diameter of a longest axis of the ellipse; and
      estimate, based on the diameter of the longest axis of the ellipse, the at least one of the one or more diametral measurements.
72. The non-transitory computer-readable medium of Embodiment 65, wherein the instructions to identify HRF in the retina of the eye of the patient further comprise instructions to classify the eye of the patient as having at least one of diabetic retinopathy (DR), diabetic macular edema (DME), age-related macular degeneration (AMD), neovascular age-related macular degeneration (nAMD), geographic atrophy (GA), macular atrophy (MA), or retinal vein occlusion (RVO).
73. The non-transitory computer-readable medium of Embodiment 65, wherein the instructions to identify HRF in the retina of the eye of the patient further comprise instructions to:
   access Early Treatment for Diabetic Retinopathy Study (ETDRS) grid mapping information identifying one or more subfields of the ETDRS grid; and
   determine, based at least in part on the ETDRS grid mapping information, one or more volumetric measurements of the identified HRF.
74. The non-transitory computer-readable medium of Embodiment 73, wherein the one or more volumetric measurements comprises one or more of a volume of HRF in the retina of the eye of the patient, an area of HRF in the retina of the eye of the patient, or a thickness of HRF in the retina of the eye of the patient.
75. The non-transitory computer-readable medium of any of Embodiments 73-74, wherein the instructions further comprise instructions to determine, based at least in part on the ETDRS grid mapping information, a volume of HRF in the retina of the eye of the patient with respect to at least one of the identified one or more subfields.
76. The non-transitory computer-readable medium of Embodiment 75, wherein the instructions to determine the volume of HRF in the retina of the eye of the patient further comprise instructions to determine a reduction in the volume of HRF in the retina of the eye of the patient.
77. The non-transitory computer-readable medium of Embodiment 75, wherein the at least one of the identified one or more subfields comprises an outer retina subfield of the ETDRS grid.
78. The non-transitory computer-readable medium of any of Embodiments 73-77, wherein the instructions further comprise instructions to determine, based at least in part on the ETDRS grid mapping information, a quantity of HRF in the retina of the eye of the patient with respect to at least one of the identified one or more subfields.
79. The non-transitory computer-readable medium of any of Embodiments 73-78, wherein the instructions further comprise instructions to:
   access an en face image of the retina of the eye of the patient, wherein the en face image is associated with the one or more OCT scans; and
   map, based at least in part on the ETDRS grid mapping information, the identified HRF to the en face image.
80. The non-transitory computer-readable medium of Embodiment 65, wherein the one or more machine-learning models comprise at least one semantic segmentation model.
81. The non-transitory computer-readable medium of Embodiment 80, wherein the at least one semantic segmentation model comprises a U-Net architecture.
82. The non-transitory computer-readable medium of Embodiment 65, wherein the instructions further comprise instructions to train the one or more machine-learning models by:
   accessing a data set of OCT scans of a retina of an eye of one or more patients, wherein the data set of OCT scans comprise sparse annotations of HRF in the retina of the eye of the one or more patients;
   partitioning the data set of OCT scans into a model-training data set and a model-validation data set;
   training, based on the model-training data set, the one or more machine-learning models to segment OCT scans to identify sets of hyperreflective entities detectable from the OCT scans, wherein the identified sets of hyperreflective entities comprise hyperreflective material (HRM); and
   evaluating the one or more machine-learning models based on the model-validation data set.
83. The non-transitory computer-readable medium of Embodiment 82, wherein the instructions further comprise instructions to identify HRF in the retina of the eye of the one or more patients based on whether one or more diametral measurements of the HRM satisfy a predetermined diametral threshold.
84. The non-transitory computer-readable medium of Embodiment 82, wherein the sparse annotations of HRF comprise a bounding geometry encompassing a plurality of instances of HRF, and wherein the instructions further comprise instructions to:
   prior to training the one or more machine-learning models, perform an adjustment of the bounding geometry by reducing a size of the bounding geometry, the size of the bounding geometry being reduced to annotate a single instance of HRF.
85. The non-transitory computer-readable medium of Embodiment 65, wherein the one or more OCT scans comprise one or more first OCT scans of the retina of the eye of the patient being captured at an initial date, wherein the identified HRF comprises a first volume of HRF, and wherein the instructions further comprise instructions to:
   access one or more second OCT scans of the retina of the eye of the patient;
   input the one or more second OCT scans into the one or more machine-learning models to segment the one or more second OCT scans to identify a second set of hyperreflective entities detectable from the one or more second OCT scans;
   determine, based on the segmented one or more second OCT scans, one or more second diametral measurements corresponding to each of the identified second set of hyperreflective entities;
   identify a second volume of HRF in the retina of the eye of the patient based on whether at least one of the one or more second diametral measurements satisfy the diametral threshold; and
   determine, based on the second volume of HRF, whether the eye of the patient is responsive to a treatment.
86. The non-transitory computer-readable medium of Embodiment 85, wherein the instructions further comprise instructions to determine, based on the second volume of HRF, a degree to which the eye of the patient is responsive to the treatment.
87. The non-transitory computer-readable medium of any of Embodiments 85-86, wherein the eye of the patient is responsive to the treatment when the second volume of HRF is less than the first volume of HRF.
88. The non-transitory computer-readable medium of any of Embodiments 85-87, wherein the treatment comprises an anti-vascular endothelial growth factor (anti-VEGF) antibody, an anti-vascular endothelial growth factor-A (anti-VEGF-A) antibody, an anti-anangiopoietin-2 (anti-Ang-2) antibody, or a combination thereof.
89. The non-transitory computer-readable medium of Embodiments 85-88, wherein the anti-VEGF-A antibody comprises faricimab-svoa.
90. The non-transitory computer-readable medium of Embodiments 85-89, wherein the anti-Ang-2 antibody comprises faricimab-svoa.
91. The non-transitory computer-readable medium of Embodiments 85-90, wherein the anti-VEGF antibody is selected from the group comprising ranibizumab, aflibercept, brolucizumab, bevacizumab, and pegaptanib sodium.
92. The non-transitory computer-readable medium of Embodiment 65, wherein the instructions further comprise instructions to identify an effective treatment regimen of an anti-vascular endothelial growth factor (anti-VEGF) antibody, an anti-anangiopoietin-2 (anti-Ang-2) antibody, or a combination thereof, to treat the eye of the patient based on a volume or a quantity of the identified HRF.
93. The non-transitory computer-readable medium of Embodiment 92, wherein the instructions to identify the effective treatment regimen further comprise instructions to identify, based on the volume or the quantity of the identified HRF, a dosage for administering the anti-VEGF antibody, the anti-Ang-2 antibody, or the combination thereof.
94. The non-transitory computer-readable medium of any of Embodiments 92-93, wherein the instructions to identify the effective treatment regimen further comprise instructions to identify, based on the volume or the quantity of the identified HRF, a schedule for administering the anti-VEGF antibody, the anti-Ang-2 antibody, or the combination thereof.
95. The non-transitory computer-readable medium of any of Embodiments 92-94, wherein the instructions to identify the effective treatment regimen further comprise instructions to identify, based on the volume or the quantity of the identified HRF, a duration for administering the anti-VEGF antibody, the anti-Ang-2 antibody, or the combination thereof.
96. The non-transitory computer-readable medium of Embodiment 85, wherein the one or more second OCT scans are captured at one or more dates selected from the group comprising approximately 0.25 months, 0.5 months, 0.75 months, 1 month, 3 months, 6 months, 9 months, 12 months, 15 months, 18 months, 21 months, 24 months, 27 months, 30 months, 33 months, or 36 months from the initial date.
97. The method of any of Embodiments 1-32, further comprising: determining whether the at least one of the one or more diametral measurements satisfy a third diametral threshold.
98. The method of Embodiment 97, wherein the third diametral threshold comprises a minimum diameter of approximately 100 microns (μm).
99. The system of any of Embodiments 33-64, further comprising: determining whether the at least one of the one or more diametral measurements satisfy a third diametral threshold.
100. The system of Embodiment 99, wherein the third diametral threshold comprises a minimum diameter of approximately 100 microns (μm).
101. The non-transitory computer-readable medium of any of Embodiments 65-96, further comprising: determining whether the at least one of the one or more diametral measurements satisfy a third diametral threshold.
102. The non-transitory computer-readable medium of Embodiment 101, wherein the third diametral threshold comprises a minimum diameter of approximately 100 microns (μm).

What is claimed is:

1. A method for identifying hyperreflective foci (HRF) in an eye of a patient, comprising, by one or more computing devices:
accessing one or more optical coherence tomography (OCT) scans of a retina of the eye of the patient;
inputting the one or more OCT scans into one or more machine-learning models trained to segment the one or more OCT scans to identify a set of hyperreflective entities detectable from the one or more OCT scans;
determining, based on the segmented one or more OCT scans, one or more diametral measurements corresponding to each of the identified set of hyperreflective entities; and
identifying hyperreflective foci (HRF) in the retina of the eye of the patient based on whether at least one of the one or more diametral measurements satisfy a diametral threshold.

2. The method of claim 1, wherein the set of hyperreflective entities comprise a set of a hyperreflective material (HRM), intraretinal hyperreflective material (IHRM), and HRF.

3. The method of claim 1, wherein identifying HRF in the retina of the eye of the patient comprises identifying a subset of the identified set of hyperreflective entities.

4. The method of claim 1, further comprising identifying intraretinal hyperreflective material (IHRM) in the retina of the eye of the patient based on whether the at least one of the one or more diametral measurements satisfy a second diametral threshold.

5. The method of claim 1, wherein the diametral threshold comprises a minimum diameter of approximately 50 microns (μm).

6. The method of claim 1, wherein determining whether the at least one of the one or more diametral measurements satisfy the diametral threshold further comprises:
for each of the identified set of hyperreflective entities:
associating an ellipse with the identified hyperreflective entity;
determining a diameter of a longest axis of the ellipse; and
estimating, based on the diameter of the longest axis of the ellipse, the at least one of the one or more diametral measurements.

7. The method of claim 1, wherein identifying HRF in the retina of the eye of the patient further comprises classifying the eye of the patient as having at least one of diabetic retinopathy (DR), diabetic macular edema (DME), age-related macular degeneration (AMD), neovascular age-related macular degeneration (nAMD), geographic atrophy (GA), macular atrophy (MA), or retinal vein occlusion (RVO).

8. The method of claim 1, wherein identifying HRF in the retina of the eye of the patient further comprises:
accessing Early Treatment for Diabetic Retinopathy Study (ETDRS) grid mapping information identifying one or more subfields of the ETDRS grid; and
determining, based at least in part on the ETDRS grid mapping information, one or more volumetric measurements of the identified HRF.

9. The method of claim 8, wherein the one or more volumetric measurements comprises one or more of a volume of HRF in the retina of the eye of the patient, an area of HRF in the retina of the eye of the patient, or a thickness of HRF in the retina of the eye of the patient.

10. The method of claim 1, wherein the one or more machine-learning models comprise at least one semantic segmentation model.

11. The method of claim 1, further comprising training the one or more machine-learning models by:
accessing a data set of OCT scans of a retina of an eye of one or more patients, wherein the data set of OCT scans comprise sparse annotations of HRF in the retina of the eye of the one or more patients;
partitioning the data set of OCT scans into a model-training data set and a model-validation data set;
training, based on the model-training data set, the one or more machine-learning models to segment OCT scans to identify sets of hyperreflective entities detectable from the OCT scans, wherein the identified sets of hyperreflective entities comprise hyperreflective material (HRM); and
evaluating the one or more machine-learning models based on the model-validation data set.

12. The method of claim 11, further comprising identifying HRF in the retina of the eye of the one or more patients based on whether one or more diametral measurements of the HRM satisfy a predetermined diametral threshold.

13. The method of claim 1, wherein the one or more OCT scans comprise one or more first OCT scans of the retina of the eye of the patient being captured at an initial date, and wherein the identified HRF comprises a first volume of HRF, the method further comprising:
accessing one or more second OCT scans of the retina of the eye of the patient;
inputting the one or more second OCT scans into the one or more machine-learning models to segment the one or more second OCT scans to identify a second set of hyperreflective entities detectable from the one or more second OCT scans;
determining, based on the segmented one or more second OCT scans, one or more second diametral measurements corresponding to each of the identified second set of hyperreflective entities;
identifying a second volume of HRF in the retina of the eye of the patient based on whether at least one of the one or more second diametral measurements satisfy the diametral threshold; and
determining, based on the second volume of HRF, whether the eye of the patient is responsive to a treatment.

14. The method of claim 13, further comprising determining, based on the second volume of HRF, a degree to which the eye of the patient is responsive to the treatment.

15. The method of claim 13, wherein the treatment comprises an anti-vascular endothelial growth factor (anti-VEGF) antibody, an anti-vascular endothelial growth factor-A (anti-VEGF-A) antibody, an anti-anangiopoietin-2 (anti-Ang-2) antibody, or a combination thereof.

16. The method of claim 1, further comprising identifying an effective treatment regimen of an anti-vascular endothelial growth factor (anti-VEGF) antibody, an anti-anangiopoietin-2 (anti-Ang-2) antibody, or a combination thereof, to treat the eye of the patient based on a volume or a quantity of the identified HRF.

17. The method of claim 16, wherein identifying the effective treatment regimen comprises identifying, based on the volume or the quantity of the identified HRF, (i) a dosage for administering the anti-VEGF antibody, the anti-Ang-2 antibody, or a combination thereof and/or (ii) a schedule for administering the anti-VEGF antibody, the anti-Ang-2 antibody, or a combination thereof.

18. The method of claim 16, wherein identifying the effective treatment regimen comprises identifying, based on the volume or the quantity of the identified HRF, a duration for administering the anti-VEGF antibody, the anti-Ang-2 antibody, or the combination thereof.

19. A system including one or more computing devices for identifying hyperreflective foci (HRF) in an eye of a patient, the one or more computing devices comprising:
one or more non-transitory computer-readable storage media including instructions; and
one or more processors coupled to the one or more storage media, the one or more processors configured to execute the instructions to:
access one or more optical coherence tomography (OCT) scans of a retina of the eye of the patient;
input the one or more OCT scans into one or more machine-learning models trained to segment the one or more OCT scans to identify a set of hyperreflective entities detectable from the one or more OCT scans;
determine, based on the segmented one or more OCT scans, one or more diametral measurements corresponding to each of the identified set of hyperreflective entities; and
identify hyperreflective foci (HRF) in the retina of the eye of the patient based on whether at least one of the one or more diametral measurements satisfy a diametral threshold.

20. A non-transitory computer-readable medium comprising instructions that, when executed by one or more processors of one or more computing devices, cause the one or more processors to:
access one or more optical coherence tomography (OCT) scans of a retina of the eye of the patient;
input the one or more OCT scans into one or more machine-learning models trained to segment the one or more OCT scans to identify a set of hyperreflective entities detectable from the one or more OCT scans;
determine, based on the segmented one or more OCT scans, one or more diametral measurements corresponding to each of the identified set of hyperreflective entities; and
identify hyperreflective foci (HRF) in the retina of the eye of the patient based on whether at least one of the one or more diametral measurements satisfy a diametral threshold.
